(19) United States
(12) Patent Application Publication
(10) Pub. No.: US 2020/0087410 A1
(43) Pub. Date: Mar. 19, 2020

(54) HUMANIZED ANTI-CD40 ANTIBODIES

(71) Applicant: Glycotope GmbH, Berlin (DE)

(72) Inventors: Steffen GOLETZ, Berlin (DE); Antje DANIELCZYK, Berlin (DE); Anja LOFFLER, Berlin (DE); Doreen WEIGELT, Berlin (DE); Patrick KEHLER, Berlin (DE)

(73) Assignee: Glycotope GmbH, Berlin (DE)

(21) Appl. No.: 16/495,528

(22) PCT Filed: Mar. 27, 2018
(86) PCT No.: PCT/EP2018/057717

§ 371 Date: Sep. 19, 2019

(30) Foreign Application Priority Data
Mar. 29, 2017 (LU) LU100151

(51) Int. Cl. C07K 16/28

(52) U.S. Cl. CPC C07K 16/2878; C07K 2317/24; C07K 2317/565; C07K 2317/14; C07K 2317/92; C07K 2317/75; C07K 2317/41; C07K 2317/52

(57) ABSTRACT

The present invention pertains to humanized anti-CD40 antibodies having antigen binding properties similar to those of the parent chimeric anti-CD40 antibody and increased Fcγ receptor binding affinities. In particular, the present invention is directed to humanized anti-CD40 antibodies which are useful in the treatment of cancer, infections and immunodeficiencies.

### FIELD OF THE INVENTION

The present invention pertains to the field of antibodies. In particular, a humanized anti-CD40 antibody showing strong antigen binding and/or Fc receptor binding is provided. In specific embodiments, the present invention is directed to humanized anti-CD40 antibodies for therapeutic and diagnostic use.

### BACKGROUND OF THE INVENTION

Today, antibodies are widely used agents in the field of medicine and research. In medicine, they find application in many different fields. For example, antibodies are used as therapeutic agents in the treatment and prophylaxis of a variety of diseases such as cancer, cardiovascular diseases, inflammatory diseases, macular degeneration, transplant rejection, multiple sclerosis, and viral infections. In these therapies, the antibody may possess therapeutic activity on its own, for example by blocking receptors or messenger molecules, thereby inhibiting their disease-relevant functions, or by recruiting and activating components of the patient's immune system.

Specific antibodies are produced by injecting an antigen into a mammal, such as a mouse, rat, rabbit, goat, sheep, or horse. Blood isolated from these animals contains polyclonal antibodies directed against said antigen in the serum. To obtain an antibody that is specific for a single epitope of an antigen, antibody-secreting lymphocytes are isolated from the animal and immortalized by fusing them with a cancer cell line, resulting in hybridoma cells. Single hybridoma cells are then isolated by dilution cloning to generate cell clones that all produce the same monoclonal antibody.

However, in therapeutic applications these monoclonal antibodies have the problem that they are derived from animal organisms and differ in their amino acid sequence from human antibodies. The human immune system hence recognizes these animal antibodies as foreign and rapidly removes them from circulation. Furthermore, systemic inflammatory effects may be caused. A solution to this problem is the replacement of certain constant parts of the monoclonal antibody with corresponding parts of a human antibody. If only the heavy and light chain constant regions are replaced, a chimeric antibody is obtained, while the additional replacement of the framework regions of the heavy and light chain variable regions results in so called humanized antibodies.

In research, purified antibodies are used in many applications. They are most commonly used to identify and locate biological molecules such as in particular proteins. The biological molecules may either be detected after they have been isolated, for example to determine their presence, concentration, integrity or size. On the other hand, they may be detected in cellular or tissue samples, for example to determine their presence or location. Furthermore, antibodies are used in isolation procedures of specific biological substances, in particular proteins, wherein the antibody specifically separates the biological substance of interest from the sample containing it.

In all these applications, a tight binding and specific recognition of the antigen is of vital importance for the antibody used. Thereby, higher activity and less cross-reactivity, in particular less adverse side effects in therapeutic applications, are obtained. However, during humanization of monoclonal antibodies, often the affinity and specificity of the engineered antibody is decreased.

An interesting and important group of antibodies are those directed against CD40, a member of the tumor necrosis factor (TNF) receptor family. CD40 is a costimulatory protein found on immune cells, in particular on antigen presenting cells. It is expressed in many cell types, including B lymphocytes, macrophages, dendritic cells, monocytes and many tumor cells, especially B cell lymphomas and solid tumors. CD40 is a transmembrane receptor and is important for activation of immune cells. The main ligand of CD40 is CD40L (CD154), which is a transmembrane protein expressed on activated T cells, in particular activated T helper cells. Recognition of an antigen presented on a B cell by a T helper cell leads to activation of the B cell only if CD40 of the B cell is bound by CD40L of the T cell. Lack of the secondary CD40-CD40L signal can lead to immune tolerance of the presented antigen. CD40 activation results in B cell proliferation, maturation, cytokine production and class switching. Likewise, activation of CD40 on monocytes and dendritic cells enhances survival and cytokine secretion and maturation of dendritic cells.

CD40 activation is also effective for antitumor responses. Tolerance of tumor antigens is prevented or reversed by CD40 activation, resulting in T cell responses against the tumor cells. Activation of CD40 can also be achieved by agonistic anti-CD40 antibodies. Hence, agonistic anti-CD40 antibodies may be used for inducing tumor clearance, in particular via activation of dendritic cells and T cell responses.

It was further demonstrated that the immunomodulatory activity of agonistic anti-CD40 antibodies is enhanced by interaction with Fcγ receptors, especially FcγRII. Not only the Fcγ receptor signaling, but in particular the crosslinking effect of receptor binding is critical for the activity of the agonistic antibodies.

A known agonistic antibody against CD40 is the monoclonal antibody ChiLob 7/4. However, this is a chimeric antibody having murine variable regions which may cause the problems of non-human antibodies discussed above. Humanization of this antibody would hence be beneficial. Unfortunately, a humanized antibody often has a lower affinity and specificity for its target antigen than the corresponding non-human or chimeric antibody. This, as the overall three-dimensional structure of the variable regions and in particular the conformation and orientation of the complementarity determining regions (CDRs) may be altered by the replacement of the framework regions.

Therefore, there is a need in the art to provide humanized antibodies, in particular humanized anti-CD40 antibodies, in particular humanized versions of ChiLob 7/4, which have an antigen binding affinity and antigen specificity similar to that of the corresponding murine or chimeric antibody.

### SUMMARY OF THE INVENTION

The present inventors have found humanized anti-CD40 antibodies having the same antigen binding affinity as the parent chimeric antibody from which they are derived. Furthermore, these humanized antibodies exhibit an enhanced Fcγ receptor binding affinity, especially with respect to FcγRII.

Therefore, in a first aspect, the present invention is directed to a humanized antibody which is capable of binding to CD40 and which comprises a heavy chain variable region, wherein the heavy chain variable region comprises the amino acid sequence of SEQ ID NO: 11, or an amino acid sequence which is at least 90% identical to the amino acid sequence of SEQ ID NO: 11.

In a second aspect, the present invention provides a nucleic acid encoding the antibody according to the invention. Furthermore, in a third aspect an expression cassette or vector comprising the nucleic acid according to the invention and a promoter operatively connected with said nucleic acid and, in a fourth aspect, a host cell comprising the nucleic acid or the expression cassette or vector according to the invention are provided.

In a fifth aspect, the present invention provides a conjugate comprising the antibody according to the invention conjugated to a further agent.

In a sixth aspect, the present invention is directed to a composition comprising the antibody according to the invention, the nucleic acid according to the invention, the expression cassette or vector according to the invention, the host cell according to the invention, or the conjugate according to the invention.

According to a seventh aspect, the invention provides the antibody, the nucleic acid, the expression cassette or vector, the host cell, the composition or the conjugate according to the invention for use in medicine, in particular in the treatment of cancer.

Other objects, features, advantages and aspects of the present invention will become apparent to those skilled in the art from the following description and appended claims. It should be understood, however, that the following description, appended claims, and specific examples, which indicate preferred embodiments of the application, are given by way of illustration only. Various changes and modifications within the spirit and scope of the disclosed invention will become readily apparent to those skilled in the art from reading the following.

### Definitions

As used herein, the following expressions are generally intended to preferably have the meanings as set forth below, except to the extent that the context in which they are used indicates otherwise.

The expression “comprise”, as used herein, besides its literal meaning also includes and specifically refers to the expressions “consist essentially of” and “consist of”. Thus, the expression “comprise” refers to embodiments wherein the subject-matter which “comprises” specifically listed elements does not comprise further elements as well as embodiments wherein the subject-matter which “comprises” specifically listed elements may and/or indeed does encompass further elements. Likewise, the expression “have” is to be understood as the expression “comprise”, also including and specifically referring to the expressions “consist essentially of” and “consist of”. The term “consist essentially of”, where possible, in particular refers to embodiments wherein the subject-matter comprises 20% or less, in particular 15% or less, 10% or less or especially 5% or less further elements in addition to the specifically listed elements of which the subject-matter consists essentially of.

The term “antibody” in particular refers to a protein comprising at least two heavy chains and two light chains connected by disulfide bonds. Each heavy chain is comprised of a heavy chain variable region (VH) and a heavy chain constant region (CH). Each light chain is comprised of a light chain variable region (VL) and a light chain constant region (CL). The heavy chain-constant region comprises three or—in the case of antibodies of the IgM- or IgE-type—four heavy chain-constant domains (CH1, CH2, CH3 and CH4) wherein the first constant domain CH1 is adjacent to the variable region and may be connected to the second constant domain CH2 by a hinge region. The light chain-constant region consists only of one constant domain. The variable regions can be further subdivided into regions of hypervariability, termed complementarity determining regions (CDRs), interspersed with regions that are more conserved, termed framework regions (FR), wherein each variable region comprises three CDRs and four FRs. The variable regions of the heavy and light chains contain a binding domain that interacts with an antigen. The heavy chain constant regions may be of any type such as γ-, δ-, α-, μ- or ε-type heavy chains. Preferably, the heavy chain of the antibody is a γ-chain. Furthermore, the light chain constant region may also be of any type such as κ- or λ-type light chains. Preferably, the light chain of the antibody is a κ-chain. The terms “γ- (δ-, α-, μ- or ε-) type heavy chain” and “κ- (λ-) type light chain” refer to antibody heavy chains or antibody light chains, respectively, which have constant region amino acid sequences derived from naturally occurring heavy or light chain constant region amino acid sequences, especially human heavy or light chain constant region amino acid sequences. In particular, the amino acid sequence of the constant domains of a γ-type (especially γ1-type) heavy chain is at least 95%, especially at least 98%, identical to the amino acid sequence of the constant domains of a human γ (especially one of the allotypes of the human γ1) antibody heavy chain. Furthermore, the amino acid sequence of the constant domain of a κ-type light chain is in particular at least 95%, especially at least 98%, identical to the amino acid sequence of the constant domain of one of the allotypes of the human κ antibody light chain. The constant regions of the antibodies may mediate the binding of the immunoglobulin to host tissues or factors, including various cells of the immune system (e.g., effector cells) and the first component (C1q) of the classical complement system. The antibody can be e.g. a humanized, human or chimeric antibody.

The antigen-binding portion of an antibody usually refers to full length or one or more fragments of an antibody that retains the ability to specifically bind to an antigen. It has been shown that the antigen-binding function of an antibody can be performed by fragments of a full-length antibody. Examples of binding fragments of an antibody include a Fab fragment, a monovalent fragment consisting of the V_{L}, V_{H}, C_{L} and C_{H1} domains; a F(ab)₂ fragment, a bivalent fragment comprising two Fab fragments, each of which binds to the same antigen, linked by a disulfide bridge at the hinge region; a Fd fragment consisting of the V_{H} and C_{H1} domains; a Fv fragment consisting of the V_{L} and V_{H} domains of a single arm of an antibody; and a dAb fragment, which consists of a V_{H} domain.

The “Fab part” of an antibody in particular refers to a part of the antibody comprising the heavy and light chain variable regions (V_{H} and V_{L}) and the first domains of the heavy and light chain constant regions (C_{H1} and C_{L}). In cases where the antibody does not comprise all of these regions, then the term “Fab part” only refers to those of the regions V_{H}, V_{L}, C_{H1} and C_{L} which are present in the antibody. Preferably, “Fab part” refers to that part of an antibody corresponding to the fragment obtained by digesting a natural antibody with papain which contains the antigen binding activity of the antibody. In particular, the Fab part of an antibody encompasses the antigen binding site or antigen binding ability thereof. Preferably, the Fab part comprises at least the V_{H} region of the antibody.

The “Fc part” of an antibody in particular refers to a part of the antibody comprising the heavy chain constant regions 2, 3 and—where applicable—4 (C_{H2}, C_{H3} and C_{H4}). In particular, the Fc part comprises two of each of these regions. In cases where the antibody does not comprise all of these regions, then the term “Fc part” only refers to those of the regions C_{H2}, C_{H3} and C_{H4} which are present in the antibody. Preferably, the Fc part comprises at least the C_{H2} region of the antibody. Preferably, “Fc part” refers to that part of an antibody corresponding to the fragment obtained by digesting a natural antibody with papain which does not contain the antigen binding activity of the antibody. In particular, the Fc part of an antibody is capable of binding to the Fc receptor and thus, e.g. comprises an Fc receptor binding site or an Fc receptor binding ability.

According to the present invention, the term “chimeric antibody” in particular refers to an antibody wherein the constant regions are derived from a human antibody or a human antibody consensus sequence, and wherein at least one and preferably both variable regions are derived from a non-human antibody, e.g. from a rodent antibody such as a mouse antibody.

According to the present invention, the term “humanized antibody” in particular refers to a non-human antibody comprising human constant regions and variable regions which amino acid sequences are modified so as to reduce the immunogenicity of the antibody when administered to the human body. An exemplary method for constructing humanized antibodies is CDR grafting, wherein the CDRs or the specificity determining residues (SDRs) of a non-human antibody are combined with human-derived framework regions. Optionally, some residues of the human framework regions may be backmutated towards the residues of the parent non-human antibody, e.g. for increasing or restoring the antigen binding affinity. Other humanization methods include, for example, resurfacing, superhumanization, and human string content optimization. In the resurfacing methods, only those residues of the non-human framework regions which are positioned at the surface of the antibody are replaced by residues present in corresponding human antibody sequences at said position. Superhumanization essentially corresponds to CDR grafting. However, while during CDR grafting the human framework regions are normally chosen based on their homology to the non-human framework regions, in superhumanization it is the similarity of the CDRs on the basis of which the human framework regions are chosen. In the human string content optimization the differences of the non-human antibody sequence to the human germline sequences is scored and then the antibody is mutated to minimize said score. Furthermore, humanized antibodies can also be obtained by empirical methods wherein large libraries of human framework regions or human antibodies are used to generate multiple antibody humanized candidates and then the most promising candidate is determined by screening methods. Also with the above-described rational approaches several humanized antibody candidates can be generated and then screened, for example for their antigen binding.

The term “human antibody”, as used herein, is intended to include antibodies having variable regions in which both the framework and CDR regions are derived from sequences of human origin.

The term “antibody”, as used herein, refers in certain embodiments to a population of antibodies of the same kind. In particular, all antibodies of the population of the antibody exhibit the features used for defining the antibody. In certain embodiments, all antibodies in the population of the antibody have the same amino acid sequence. Reference to a specific kind of antibody, such as an anti-CD40 antibody, in particular refers to a population of this kind of antibody.

The term “antibody” as used herein also includes fragments and derivatives of said antibody. A “fragment or derivative” of an antibody in particular is a protein or glycoprotein which is derived from said antibody and is capable of binding to the same antigen, in particular to the same epitope as the antibody. Thus, a fragment or derivative of an antibody herein generally refers to a functional fragment or derivative. In particularly preferred embodiments, the fragment or derivative of an antibody comprises a heavy chain variable region. It has been shown that the antigen-binding function of an antibody can be performed by fragments of a full-length antibody or derivatives thereof. Examples of fragments of an antibody include (i) Fab fragments, monovalent fragments consisting of the variable region and the first constant domain of each the heavy and the light chain; (ii) F(ab)₂ fragments, bivalent fragments comprising two Fab fragments linked by a disulfide bridge at the hinge region; (iii) Fd fragments consisting of the variable region and the first constant domain CH1 of the heavy chain; (iv) Fv fragments consisting of the heavy chain and light chain variable region of a single arm of an antibody; (v) scFv fragments, Fv fragments consisting of a single polypeptide chain; (vi) (Fv)₂ fragments consisting of two Fv fragments covalently linked together; (vii) a heavy chain variable domain; and (viii) multibodies consisting of a heavy chain variable region and a light chain variable region covalently linked together in such a manner that association of the heavy chain and light chain variable regions can only occur intermolecular but not intramolecular. Derivatives of an antibody in particular include antibodies which bind to the same antigen as the parent antibody, but which have a different amino acid sequence than the parent antibody from which it is derived. These antibody fragments and derivatives are obtained using conventional techniques known to those with skill in the art.

A target amino acid sequence is “derived” from or “corresponds” to a reference amino acid sequence if the target amino acid sequence shares a homology or identity over its entire length with a corresponding part of the reference amino acid sequence of at least 75%, more preferably at least 80%, at least 85%, at least 90%, at least 93%, at least 95%, at least 97%, at least 98% or at least 99%. The “corresponding part” means that, for example, framework region 1 of a heavy chain variable region (FRH1) of a target antibody corresponds to framework region 1 of the heavy chain variable region of the reference antibody. In particular embodiments, a target amino acid sequence which is “derived” from or “corresponds” to a reference amino acid sequence is 100% homologous, or in particular 100% identical, over its entire length with a corresponding part of the reference amino acid sequence. A “homology” or “identity” of an amino acid sequence or nucleotide sequence is preferably determined according to the invention over the entire length of the reference sequence or over the entire length of the corresponding part of the reference sequence which corresponds to the sequence which homology or identity is defined. An antibody derived from a parent antibody which is defined by one or more amino acid sequences, such as specific CDR sequences or specific variable region sequences, in particular is an antibody having amino acid sequences, such as CDR sequences or variable region sequences, which are at least 75%, preferably at least 80%, at least 85%, at least 90%, at least 93%, at least 95%, at least 97%, at least 98% or at least 99% homologous or identical, especially identical, to the respective amino acid sequences of the parent antibody. In certain embodiments, the antibody derived from (i.e. derivative of) a parent antibody comprises the same CDR sequences as the parent antibody, but differs in the remaining sequences of the variable regions.

The term “antibody” as used herein also refers to multivalent and multispecific antibodies, i.e. antibody constructs which have more than two binding sites each binding to the same epitope and antibody constructs which have one or more binding sites binding to a first epitope and one or more binding sites binding to a second epitope, and optionally even further binding sites binding to further epitopes.

“Specific binding” preferably means that an agent such as an antibody binds stronger to a target such as an epitope for which it is specific compared to the binding to another target. An agent binds stronger to a first target compared to a second target if it binds to the first target with a dissociation constant (K_{d}) which is lower than the dissociation constant for the second target. Preferably the dissociation constant for the target to which the agent binds specifically is more than 100-fold, 200-fold, 500-fold or more than 1000-fold lower than the dissociation constant for the target to which the agent does not bind specifically. Furthermore, the term “specific binding” in particular indicates a binding affinity between the binding partners with an affinity constant Kₐ of at least 10⁶ M⁻¹, preferably at least 10⁷ M⁻¹, more preferably at least 10⁸ M⁻¹. An antibody specific for a certain antigen in particular refers to an antibody which is capable of binding to said antigen with an affinity having a Kₐ of at least 10⁶ M⁻¹, preferably at least 10⁷ M⁻¹, more preferably at least 10⁸ M⁻¹. For example, the term “anti-CD40 antibody” refers to an antibody specifically binding CD40 and preferably is capable of binding to CD40 with an affinity having a Kₐ of at least 10⁶ M⁻¹, preferably at least 10⁷ M⁻¹, more preferably at least 10⁸ M⁻¹.

The term “ChiLob 7/4” as used herein in particular refers to a human/mouse chimeric antibody having the heavy chain and light chain variable region amino acid sequences of SEQ ID NOs: 22 and 23, respectively.

The term “agonistic antibody” refers to an antibody which, upon binding to its antigen, is capable of activating its antigen. If the antigen is a receptor, the agonistic antibody may mimic ligand binding, e.g. via binding to the ligand binding domain of the receptor or via binding to two receptor molecules, thereby initiating dimer formation.

The term “CD40” according to the present invention in particular refers to human CD40, also known as TNF receptor super family member 5 (TNFRSF5). CD40 is a member of the tumor necrosis factor receptor superfamily. It is a transmembrane protein comprising an extracellular ligand binding domain, a membrane-spanning domain and an intracellular domain. Upon binding of a ligand, in particular CD40L, CD40 is activated, resulting in intracellular downstream signaling.

The term “sialic acid” in particular refers to any N- or O-substituted derivatives of neuraminic acid. It may refer to both 5-N-acetylneuraminic acid and 5-N-glycolylneuraminic acid, but preferably only refers to 5-N-acetylneuraminic acid. The sialic acid, in particular the 5-N-acetylneuraminic acid preferably is attached to a carbohydrate chain via a 2,3- or 2,6-linkage. Preferably, in the antibodies described herein both 2,3- as well as 2,6-coupled sialic acids are present.

A “relative amount of glycans” according to the invention refers to a specific percentage or percentage range of the glycans attached to the antibodies of an antibody preparation or in a composition comprising antibodies, respectively. In particular, the relative amount of glycans refers to a specific percentage or percentage range of all glycans comprised in the antibodies and thus, attached to the polypeptide chains of the antibodies in an antibody preparation or in a composition comprising antibodies. 100% of the glycans refers to all glycans attached to the antibodies of the antibody preparation or in a composition comprising antibodies, respectively. For example, a relative amount of glycans carrying bisecting GlcNAc of 10% refers to a composition comprising antibodies wherein 10% of all glycans comprised in the antibodies and thus, attached to the antibody polypeptide chains in said composition comprise a bisecting GlcNAc residue while 90% of all glycans comprised in the antibodies and thus, attached to the antibody polypeptide chains in said composition do not comprise a bisecting GlcNAc residue. The corresponding reference amount of glycans representing 100% may either be all glycan structures attached to the antibodies in the composition, or all N-glycans, i.e. all glycan structures attached to an asparagine residue of the antibodies in the composition, or all complex-type glycans. The reference group of glycan structures generally is explicitly indicated or directly derivable from the circumstances by the skilled person.

The term “N-glycosylation” refers to all glycans attached to asparagine residues of the polypeptide chain of a protein. These asparagine residues generally are part of N-glycosylation sites having the amino acid sequence Asn-Xaa-Ser/Thr, wherein Xaa may be any amino acid except for proline. Likewise, “N-glycans” are glycans attached to asparagine residues of a polypeptide chain. The terms “glycan”, “glycan structure”, “carbohydrate”, “carbohydrate chain” and “carbohydrate structure” are generally used synonymously herein. N-glycans generally have a common core structure consisting of two N-acetylglucosamine (GlcNAc) residues and three mannose residues, having the structure Manα1,6-(Manα1,3-)Manβ1,4-GlcNAcβ1,4-GlcNAcβ1-Asn with Asn being the asparagine residue of the polypeptide chain. N-glycans are subdivided into three different types, namely complex-type glycans, hybrid-type glycans and high mannose-type glycans.

The numbers given herein, in particular the relative amounts of a specific glycosylation property, are preferably to be understood as approximate numbers. In particular, the numbers preferably may be up to 10% higher and/or lower, in particular up to 9%, 8%, 7%, 6%, 5%, 4%, 3%, 2% or 1% higher and/or lower.

In a “conjugate” two or more compounds are linked together. In certain embodiments, at least some of the properties from each compound are retained in the conjugate. Linking may be achieved by a covalent or non-covalent bond. Preferably, the compounds of the conjugate are linked via a covalent bond. The different compounds of a conjugate may be directly bound to each other via one or more covalent bonds between atoms of the compounds. Alternatively, the compounds may be bound to each other via a chemical moiety such as a linker molecule wherein the linker is covalently attached to atoms of the compounds. If the conjugate is composed of more than two compounds, then these compounds may, for example, be linked in a chain conformation, one compound attached to the next compound, or several compounds each may be attached to one central compound.

The term “nucleic acid” includes single-stranded and double-stranded nucleic acids and ribonucleic acids as well as deoxyribonucleic acids. It may comprise naturally occurring as well as synthetic nucleotides and can be naturally or synthetically modified, for example by methylation, 5′- and/or 3′-capping.

The term “expression cassette” in particular refers to a nucleic acid construct which is capable of enabling and regulating the expression of a coding nucleic acid sequence introduced therein. An expression cassette may comprise promoters, ribosome binding sites, enhancers and other control elements which regulate transcription of a gene or translation of an mRNA. The exact structure of expression cassette may vary as a function of the species or cell type, but generally comprises 5′-untranscribed and 5′- and 3′-untranslated sequences which are involved in initiation of transcription and translation, respectively, such as TATA box, capping sequence, CAAT sequence, and the like. More specifically, 5′-untranscribed expression control sequences comprise a promoter region which includes a promoter sequence for transcriptional control of the operatively connected nucleic acid. Expression cassettes may also comprise enhancer sequences or upstream activator sequences.

According to the invention, the term “promoter” refers to a nucleic acid sequence which is located upstream (5′) of the nucleic acid sequence which is to be expressed and controls expression of the sequence by providing a recognition and binding site for RNA-polymerases. The “promoter” may include further recognition and binding sites for further factors which are involved in the regulation of transcription of a gene. A promoter may control the transcription of a prokaryotic or eukaryotic gene. Furthermore, a promoter may be “inducible”, i.e. initiate transcription in response to an inducing agent, or may be “constitutive” if transcription is not controlled by an inducing agent. A gene which is under the control of an inducible promoter is not expressed or only expressed to a small extent if an inducing agent is absent. In the presence of the inducing agent the gene is switched on or the level of transcription is increased. This is mediated, in general, by binding of a specific transcription factor.

The term “vector” is used here in its most general meaning and comprises any intermediary vehicle for a nucleic acid which enables said nucleic acid, for example, to be introduced into prokaryotic and/or eukaryotic cells and, where appropriate, to be integrated into a genome. Vectors of this kind are preferably replicated and/or expressed in the cells. Vectors comprise plasmids, phagemids, bacteriophages or viral genomes. The term “plasmid” as used herein generally relates to a construct of extrachromosomal genetic material, usually a circular DNA duplex, which can replicate independently of chromosomal DNA.

According to the invention, the term “host cell” relates to any cell which can be transformed or transfected with an exogenous nucleic acid. The term “host cells” comprises according to the invention prokaryotic (e.g. E. coli) or eukaryotic cells (e.g. mammalian cells, in particular human cells, yeast cells and insect cells). Particular preference is given to mammalian cells such as cells from humans, mice, hamsters, pigs, goats, or primates. The cells may be derived from a multiplicity of tissue types and comprise primary cells and cell lines. A nucleic acid may be present in the host cell in the form of a single copy or of two or more copies and, in one embodiment, is expressed in the host cell.

The term “patient” means according to the invention a human being, a nonhuman primate or another animal, in particular a mammal such as a cow, horse, pig, sheep, goat, dog, cat or a rodent such as a mouse and rat. In a particularly preferred embodiment, the patient is a human being.

The term “cancer” according to the invention in particular comprises leukemias, seminomas, melanomas, teratomas, lymphomas, neuroblastomas, gliomas, rectal cancer, endometrial cancer, kidney cancer, adrenal cancer, thyroid cancer, blood cancer, skin cancer, cancer of the brain, cervical cancer, intestinal cancer, liver cancer, colon cancer, stomach cancer, intestine cancer, head and neck cancer, gastrointestinal cancer, lymph node cancer, esophagus cancer, colorectal cancer, pancreas cancer, ear, nose and throat (ENT) cancer, breast cancer, prostate cancer, cancer of the uterus, ovarian cancer and lung cancer and the metastases thereof. The term cancer according to the invention also comprises cancer metastases.

By “tumor” is meant a group of cells or tissue that is formed by misregulated cellular proliferation. Tumors may show partial or complete lack of structural organization and functional coordination with the normal tissue, and usually form a distinct mass of tissue, which may be either benign or malignant.

By “metastasis” is meant the spread of cancer cells from its original site to another part of the body. The formation of metastasis is a very complex process and normally involves detachment of cancer cells from a primary tumor, entering the body circulation and settling down to grow within normal tissues elsewhere in the body. When tumor cells metastasize, the new tumor is called a secondary or metastatic tumor, and its cells normally resemble those in the original tumor. This means, for example, that, if breast cancer metastasizes to the lungs, the secondary tumor is made up of abnormal breast cells, not of abnormal lung cells. The tumor in the lung is then called metastatic breast cancer, not lung cancer.

The term “pharmaceutical composition” particularly refers to a composition suitable for administering to a human or animal, i.e., a composition containing components which are pharmaceutically acceptable. Preferably, a pharmaceutical composition comprises an active compound or a salt or prodrug thereof together with a carrier, diluent or pharmaceutical excipient such as buffer, preservative and tonicity modifier.

### DETAILED DESCRIPTION OF THE INVENTION

The present invention is based on the development of humanized anti-CD40 antibodies having antigen binding properties similar to those of the corresponding chimeric antibody. Furthermore, the humanized sequences provide the antibody with a higher Fcγ receptor binding affinity. In particular binding to FcγRII was increased compared to the parent chimeric variant. It was shown in the art that strong binding to Fcγ receptors, especially FcγRII, is critical for agonistic anti-CD40 antibodies and their activation of a T cell response.

In view of these findings, the present invention provides a humanized antibody which is capable of binding to CD40 and which comprises a heavy chain variable region, wherein the heavy chain variable region comprises the amino acid sequence of SEQ ID NO: 11, or an amino acid sequence which is at least 90% identical to the amino acid sequence of SEQ ID NO: 11.

The humanized antibody in particular is capable to bind to human CD40, especially the extracellular region of human CD40. In preferred embodiments, the humanized antibody is an agonistic antibody so that binding of the humanized antibody to CD40 activates CD40 signaling. In particular, the humanized antibody specifically binds CD40.

Furthermore, the humanized antibody may exhibit antigen binding properties similar to those of a reference antibody comprising a heavy chain variable region with the amino acid sequence of SEQ ID NO: 22 and a light chain variable region with the amino acid sequence of SEQ ID NO: 23. Preferably, the reference antibody is the human/mouse chimeric antibody ChiLob 7/4. In particular, the humanized antibody according to the invention may specifically bind to the same antigen, preferably the same epitope, as the reference antibody, and may preferably bind to said antigen or epitope, respectively, with a comparable affinity. That is, the humanized antibody preferably binds to the antigen or epitope with an affinity having a dissociation constant which is at most 1000-fold higher than that of the reference antibody, more preferably at most 200-fold higher, at most 100-fold higher, at most 20-fold higher or at most 10-fold higher. Most preferably, the dissociation constant is about the same as that of the reference antibody, in particular being no more than 2-fold higher. Moreover, the humanized antibody preferably shows cross-specificity with the reference antibody comprising a heavy chain variable region with the amino acid sequence of SEQ ID NO: 22 and a light chain variable region with the amino acid sequence of SEQ ID NO: 23. In particular, the humanized antibody is able to block the binding of the reference antibody to CD40 if present in a high enough concentration. This may be possible if the binding of the reference antibody to CD40 is hindered when the humanized antibody according to the invention is already bound to the antigen CD40.

In certain embodiments, the heavy chain variable region comprises an amino acid sequence which is at least 93% identical to the amino acid sequence of SEQ ID NO: 11. Especially, the heavy chain variable region comprises an amino acid sequence which is at least 95%, in particular at least 98% identical to the amino acid sequence of SEQ ID NO: 11.

In specific embodiments, the heavy chain variable region of the humanized antibody comprises the complementarity determining regions CDR-H1 having the amino acid sequence of SEQ ID NO: 12, CDR-H2 having the amino acid sequence of SEQ ID NO: 13, and CDR-H3 having the amino acid sequence of SEQ ID NO: 14. In particular, the heavy chain variable region comprises an amino acid sequence which is at least 90% identical to the amino acid sequence of SEQ ID NO: 11 and in addition has three specific CDRs having the amino acid sequences of SEQ ID NOs: 12, 13 and 14. Hence, any sequence deviations to SEQ ID NO: 11 are located in the framework regions, but not in the CDRs.

Specifically, the humanized antibody may comprise a heavy chain variable region having an amino acid sequence according to any one of SEQ ID NOs: 1 to 10. In particular, the heavy chain variable region has the amino acid sequence according to any one of SEQ ID NOs: 6 to 10, especially SEQ ID NO: 6 or 10, preferably SEQ ID NO: 10. In specific embodiments, the heavy chain variable region comprises an amino acid sequence which is at least 90% identical to the amino acid sequence of SEQ ID NO: 10, especially, at least 93%, at least 95%, or in particular at least 98% identical to the amino acid sequence of SEQ ID NO: 10.

In certain embodiments, the humanized antibody comprises a light chain variable region, wherein the light chain variable region comprises the amino acid sequence of SEQ ID NO: 18, or an amino acid sequence which is at least 90% identical to the amino acid sequence of SEQ ID NO: 18.

In certain embodiments, the light chain variable region comprises an amino acid sequence which is at least 93% identical to the amino acid sequence of SEQ ID NO: 18. Especially, the light chain variable region comprises an amino acid sequence which is at least 95%, in particular at least 98% identical to the amino acid sequence of SEQ ID NO: 18.

In specific embodiments, the light chain variable region of the humanized antibody comprises the complementarity determining regions CDR-L1 having the amino acid sequence of SEQ ID NO: 19, CDR-L2 having the amino acid sequence of SEQ ID NO: 20, and CDR-L3 having the amino acid sequence of SEQ ID NO: 21. In particular, the light chain variable region comprises an amino acid sequence which is at least 90% identical to the amino acid sequence of SEQ ID NO: 18 and in addition has three specific CDRs having the amino acid sequences of SEQ ID NOs: 19, 20 and 21. Hence, any sequence deviations to SEQ ID NO: 18 are located in the framework regions, but not in the CDRs.

Specifically, the humanized antibody may comprise a light chain variable region having an amino acid sequence according to any one of SEQ ID NOs: 15 to 17. In particular, the light chain variable region has the amino acid sequence according to SEQ ID NO: 15 or 16, especially SEQ ID NO: 16. In specific embodiments, the light chain variable region comprises an amino acid sequence which is at least 90% identical to the amino acid sequence of SEQ ID NO: 16, especially, at least 93%, at least 95%, or in particular at least 98% identical to the amino acid sequence of SEQ ID NO: 16.

In specific embodiments, the humanized antibody has a heavy chain variable region which comprises an amino acid sequence which is at least 90% identical to the amino acid sequence of SEQ ID NO: 11 and in addition has three specific CDRs having the amino acid sequences of SEQ ID NOs: 12, 13 and 14; and a light chain variable region which comprises an amino acid sequence which is at least 90% identical to the amino acid sequence of SEQ ID NO: 18 and in addition has three specific CDRs having the amino acid sequences of SEQ ID NOs: 19, 20 and 21. In certain preferred embodiments, the humanized antibody comprises a heavy chain variable region comprising the amino acid sequence of SEQ ID NO: 10 and a light chain variable region comprising the amino acid sequence of SEQ ID NO: 16.

In preferred embodiments, the humanized antibody comprises an Fc region. The humanized antibody may especially be a whole antibody. The humanized antibody may be of any isotype, and in particular is an IgG-type antibody, especially IgG1, IgG2 or IgG4. In specific embodiments, the humanized antibody is IgG1-type or IgG2-type antibody.

In certain embodiments, the humanized anti-CD40 antibody has a high pI value. The pI value refers to the pH value at which a molecule, e.g. an anti-CD40 antibody, is neutrally charged, i.e. it has the same number of positive charges and negative charges. In particular, the humanized antibody may have a pI value of 8.0 or higher, especially 8.1 or higher or 8.2 or higher. In specific embodiments, the pI value of the humanized antibody is higher than that of an antibody having the same constant regions and wherein the heavy chain variable region has the amino acid sequence of SEQ ID NO: 22 and the light chain variable region has the amino acid sequence of SEQ ID NO: 23. Especially, the pI value is at least 0.4 units higher, in particular at least 0.5 units higher, or 0.6 units higher.

The humanized antibody in particular is capable of binding to one or more human Fcγ receptors, especially human Fcγ receptor IIA and/or human Fcγ receptor IIB. In certain embodiments, the humanized antibody has a binding affinity towards human Fcγ receptor IIA and/or human Fcγ receptor IIB which is stronger than that of an antibody having the same constant regions and wherein the heavy chain variable region has the amino acid sequence of SEQ ID NO: 22 and the light chain variable region has the amino acid sequence of SEQ ID NO: 23.

In certain embodiment, the humanized anti-CD40 antibody is glycosylated, especially N-glycosylated. In particular, the humanized antibody has a glycosylation site in the second constant domain of the heavy chain (CH2). An antibody normally has two heavy chains having identical amino acid sequences. Hence, the humanized antibody preferably has at least two glycosylation sites, one in each of its two CH2 domains. This glycosylation site in particular is at an amino acid position corresponding to amino acid position 297 of the heavy chain according to the Kabat numbering and has the amino acid sequence motive Asn Xaa Ser/Thr wherein Xaa may be any amino acid except proline. The N-linked glycosylation at Asn297 is conserved in mammalian IgGs as well as in homologous regions of other antibody isotypes. Due to optional additional amino acids which may be present in the variable region or other sequence modifications, the actual position of this conserved glycosylation site may vary in the amino acid sequence of the antibody. Preferably, the glycans attached to the humanized antibody are biantennary complex type N-linked carbohydrate structures, preferably comprising at least the following structure:
Asn-GlcNAc-GlcNAc-Man-(Man-GlcNAc)₂

wherein Asn is the asparagine residue of the polypeptide portion of the antibody; GlcNAc is N-acetylglucosamine and Man is mannose. The terminal GlcNAc residues may further carry a galactose residue, which optionally may carry a sialic acid residue. A further GlcNAc residue (named bisecting GlcNAc) may be attached to the Man nearest to the polypeptide. A fucose may be bound to the GlcNAc attached to the Asn.

In preferred embodiments, the humanized anti-CD40 antibody does not comprise N-glycolyl neuraminic acids (NeuGc) or detectable amounts of NeuGc. Furthermore, the humanized antibody preferably also does not comprise Galili epitopes (Galα1,3-Gal structures) or detectable amounts of the Galili epitope. In particular, the relative amount of glycans carrying NeuGc and/or Galα1,3-Gal structures is less than 0.1% or even less than 0.02% of the total amount of glycans attached to the Fc part of the humanized antibodies in the antibody population.

In particular, the humanized anti-CD40 antibody has a human glycosylation pattern. Due to these glycosylation properties, foreign immunogenic non-human structures which induce side effects are absent which means that unwanted side effects or disadvantages known to be caused by certain foreign sugar structures such as the immunogenic non-human sialic acids (NeuGc) or the Galili epitope (Gal-Gal structures), both known for rodent production systems, or other structures like immunogenic high-mannose structures as known from e.g. yeast systems are avoided.

In specific embodiments, the humanized antibody comprises a glycosylation pattern at the Fc region having a detectable amount of glycans carrying a bisecting GlcNAc residue. In particular, the relative amount of glycans carrying a bisecting GlcNAc residue is at least 0.1%, especially at least 0.2% or at least 0.5% of the total amount of glycans attached to the Fc glycosylation sites of the antibody in a composition. Furthermore, in certain embodiments the glycosylation pattern at the Fc region comprises a relative amount of glycans carrying at least one sialic acid residue of at least 2% of the total amount of glycans attached to the Fc glycosylation sites of the antibody in a composition. In particular, the relative amount of glycans carrying at least one sialic acid residue is at least 3%, especially at least 4% or at least 5% of the total amount of glycans attached to the Fc glycosylation sites of the antibody in a composition.

The humanized anti-CD40 antibody may have a glycosylation pattern at the Fc region having a high amount of core fucose or a low amount of core fucose. A reduced amount of fucosylation at the Fc region increases the ability of the antibody to induce ADCC. In particular, the binding affinity to FcγRIIIa is increased by decreasing the amount of fucosylation at the Fc region. In certain embodiments, the relative amount of glycans carrying a core fucose residue is 20% or less, especially 15% or less or 10% or less of the total amount of glycans attached to the Fc glycosylation sites of the antibody in a composition. In alternative embodiments, the relative amount of glycans carrying a core fucose residue is at least 60%, especially at least 65% or at least 70% of the total amount of glycans attached to the Fc glycosylation sites of the antibody in a composition.

The humanized anti-CD40 antibody is preferably recombinantly produced in a host cell. Hence, the humanized antibody in particular is a monoclonal antibody. The host cell used for the production of the humanized antibody may be any host cells which can be used for antibody production. Suitable host cells are in particular eukaryotic host cells, especially mammalian host cells. Exemplary host cells include yeast cells such as Pichia pastoris cell lines, insect cells such as SF9 and SF21 cell lines, plant cells, bird cells such as EB66 duck cell lines, rodent cells such as CHO, NS0, SP2/0 and YB2/0 cell lines, and human cells such as HEK293, PER.C6, CAP, CAP-T, AGE1.HN, Mutz-3 and KG1 cell lines.

In certain embodiments, the humanized anti-CD40 antibody is produced recombinantly in a human blood cell line, in particular in a human myeloid leukemia cell line. Preferred human cell lines which can be used for production of the anti-CD40 antibody as well as suitable production procedures are described in WO 2008/028686 A2. In a specific embodiment, the humanized anti-CD40 antibody is obtained by expression in a human myeloid leukemia cell line selected from the group consisting of NM-H9D8, NM-H9D8-E6 and NM-H9D8-E6Q12. These cell lines were deposited under the accession numbers DSM ACC2806 (NM-H9D8; deposited on Sep. 15, 2006), DSM ACC2807 (NM-H9D8-E6; deposited on Oct. 5, 2006) and DSM ACC2856 (NM-H9D8-E6Q12; deposited on Aug. 8, 2007) according to the requirements of the Budapest Treaty at the Deutsche Sammlung von Mikroorganismen and Zellkulturen (DSMZ), Inhoffenstrafße 7B, 38124 Braunschweig (DE) by Glycotope GmbH, Robert-Rössle-Str. 10, 13125 Berlin (DE). NM-H9D8 cells provide a glycosylation pattern with a high degree of sialylation, a high degree of bisecting GlycNAc, a high degree of galactosylation and a high degree of fucosylation. NM-H9D8-E6 and NM-H9D8-E6Q12 cells provide a glycosylation pattern similar to that of NM-H9D8 cells, except that the degree of fucosylation is very low. Other suitable cell lines include K562, a human myeloid leukemia cell line present in the American Type Culture Collection (ATCC CCL-243), as well as cell lines derived from the aforementioned.

In specific embodiments, the humanized anti-CD40 antibody is provided as conjugate comprising the antibody conjugated to a further agent such as a detectable marker or a therapeutically active substance. The humanized antibody can be conjugated to one or more further agents. If more than one further agent is present in the conjugate, these further agents may be identical or different, and in particular are all identical. Conjugation of the further agent to the humanized antibody can be achieved using any methods known in the art. The further agent may be covalently, in particular by fusion or chemical coupling, or non-covalently attached to the antibody. In certain embodiments, the further agent is covalently attached to the humanized antibody, especially via a linker moiety. The linker moiety may be any chemical entity suitable for attaching the further agent to the humanized antibody.

The further agent preferably is useful in therapy, diagnosis, prognosis and/or monitoring of a disease, in particular cancer. For example, the further agent may be selected from the group consisting of radionuclides, chemotherapeutic agents, antibodies, bispecific antibodies or antibody fragments, in particular those of different species and/or different specificity than the humanized anti-CD40 antibody, enzymes, interaction domains, detectable labels, toxins, cytolytic components, immunomodulators, immunoeffectors, cytokines, chemokines, MHC class I or class II antigens, and liposomes.

In a further aspect, the present invention provides a nucleic acid encoding the humanized anti-CD40 antibody. The nucleic acid sequence of said nucleic acid may have any nucleotide sequence suitable for encoding the antibody. However, preferably the nucleic acid sequence is at least partially adapted to the specific codon usage of the host cell or organism in which the nucleic acid is to be expressed, in particular the human codon usage. The nucleic acid may be double-stranded or single-stranded DNA or RNA, preferably double-stranded DNA such as cDNA or single-stranded RNA such as mRNA. It may be one consecutive nucleic acid molecule or it may be composed of several nucleic acid molecules, each coding for a different part of the humanized antibody.

If the humanized antibody is composed of more than one different amino acid chain, such as a light chain and a heavy chain, the nucleic acid may, for example, be a single nucleic acid molecule containing several coding regions each coding for one of the amino acid chains of the antibody, preferably separated by regulatory elements such as IRES elements in order to generate separate amino acid chains, or the nucleic acid may be composed of several nucleic acid molecules wherein each nucleic acid molecule comprises one or more coding regions each coding for one of the amino acid chains of the antibody. In addition to the coding regions encoding the humanized antibody, the nucleic acid may also comprise further nucleic acid sequences or other modifications which, for example, may code for other proteins, may influence the transcription and/or translation of the coding region(s), may influence the stability or other physical or chemical properties of the nucleic acid, or may have no function at all.

In a further aspect, the present invention provides an expression cassette or vector comprising a nucleic acid according to the invention and a promoter operatively connected with said nucleic acid. In addition, the expression cassette or vector may comprise further elements, in particular elements which are capable of influencing and/or regulating the transcription and/or translation of the nucleic acid, the amplification and/or reproduction of the expression cassette or vector, the integration of the expression cassette or vector into the genome of a host cell, and/or the copy number of the expression cassette or vector in a host cell. Suitable expression cassettes and vectors comprising respective expression cassettes for expressing antibodies are well known in the prior art and thus, need no further description here.

Furthermore, the present invention provides a host cell comprising the nucleic acid according to the invention or the expression cassette or vector according to the invention. The host cell may be any host cell. It may be an isolated cell or a cell comprised in a tissue. Preferably, the host cell is a cultured cell, in particular a primary cell or a cell of an established cell line, preferably a tumor-derived cell. Preferably, it is a bacterial cell such as E. coli, a yeast cell such as a Saccharomyces cell, in particular S. cerevisiae, an insect cell such as a Sf9 cell, or a mammalian cell, in particular a human cell such as a tumor-derived human cell, a hamster cell such as CHO, or a primate cell. In a preferred embodiment of the invention the host cell is derived from human myeloid leukaemia cells. Preferably, it is selected from the following cells or cell lines: K562, KG1, MUTZ-3 or a cell or cell line derived therefrom, or a mixture of cells or cell lines comprising at least one of those aforementioned cells. The host cell is preferably selected from the group consisting of NM-H9D8, NM-H9D8-E6, NM H9D8-E6Q12, and a cell or cell line derived from anyone of said host cells, or a mixture of cells or cell lines comprising at least one of those aforementioned cells. These cell lines and their properties are described in detail in the PCT-application WO 2008/028686 A2. In preferred embodiments, the host cell is optimized for expression of glycoproteins, in particular antibodies, having a specific glycosylation pattern. Preferably, the codon usage in the coding region of the nucleic acid according to the invention and/or the promoter and the further elements of the expression cassette or vector are compatible with and, more preferably, optimized for the type of host cell used. Preferably, the humanized antibody is produced by a host cell or cell line as described above.

In another aspect, the present invention provides a composition comprising the humanized antibody, the nucleic acid, the expression cassette or vector, the host cell, or the conjugate. The composition may also contain more than one of these components. Furthermore, the composition may comprise one or more further components selected from the group consisting of solvents, diluents, and excipients Preferably, the composition is a pharmaceutical composition. In this embodiment, the components of the composition preferably are all pharmaceutically acceptable. The composition may be a solid or fluid composition, in particular a—preferably aqueous—solution, emulsion or suspension or a lyophilized powder.

The humanized anti-CD40 antibody or the conjugate thereof in particular is useful in medicine, in particular in therapy, diagnosis, prognosis and/or monitoring of a disease, in particular a disease as described herein, preferably cancer, infections and immunodeficiencies. Therefore, in a further aspect, the invention provides the humanized antibody, the nucleic acid, the expression cassette or vector, the host cell, the conjugate, or the composition for use in medicine. Preferably, the use in medicine is a use in the treatment, prognosis, diagnosis and/or monitoring of a disease such as, for example, diseases associated with abnormal cell growth such as cancer, infections such as bacterial, viral, fungal or parasitic infections, and diseases associated with a reduce immune activity such as immunodeficiencies. In a preferred embodiment, the disease is cancer. Preferably the cancer is selected from the group consisting of ovarian cancer, breast cancer, lung cancer, pancreatic cancer, leukemia and lymphoma, especially chronic lymphatic leukemia, non-Hodgkin lymphoma, diffuse large B-cell lymphoma, B cell malignancies, multiple melanoma, follicular lymphoma and Hodgkin lymphoma.

In certain embodiments, the disease to be treated is a disease associated with abnormal cell growth such as cancer. The cancer may be CD40 positive or CD40 negative. Independent of the CD40 expression of the cancer cells, the humanized anti-CD40 antibody may be used in the therapy of said cancer in order to induce or enhance an immune response against the cancer cells. In specific embodiments, the humanized anti-CD40 antibody is used in combination with another anti-cancer therapeutic agent. Said further therapeutic agent may be any known anti-cancer drug and in particular may be an antibody against a cancer antigen. Suitable antibodies for combination with the humanized anti-CD40 antibody include anti-EGFR antibodies such as cetuximab (Erbitux), tomuzotuximab, panitumomab (Vectibix) and nimotuzumab (Theraloc), anti-HER2 antibodies such as trastuzumab (Herceptin), timigutuzumab and pertuzumab; anti-VEGF antibodies such as bevacizumab (Avastin) and vanuzizumab; anti-CD52 antibodies such as alemtuzumab (Campath); anti-CD30 antibodies such as brentuximab (Adcetris); anti-CD33 antibodies such as gemtuzumab (Mylotarg); anti-CD20 antibodies such as rituximab (Rituxan, Mabthera), tositumomab (Bexxar) and ibritumomab (Zevalin); anti-TF antibodies such as KaroMab as disclosed, for example, in WO 2004/050707, anti-MUC1 antibodies such as pankomab (gatipotuzumab), as disclosed, for example, in WO 2004/065423 and WO 2011/012309, anti-CTLA4 antibodies such as ipilimumab and tremelimumab, anti-PD1/PD-L1 antibodies such as pembrolizumab, nivolumab, atezolizumab, and avelumab, antibodies against TNF and TNFR superfamily members such as urelumab, MED16469, TRX518, and varilumab; CSF1R antibodies such as emactuzumab; anti-B7-H3 antibodies such as enoblituzumab; anti-LAG3 antibodies; anti-4-1 BB antibodies; anti-ICOS antibodies; and anti-OX-40 antibodies.

Further anti-cancer therapeutic agents which may be combined with the humanized anti-CD40 antibody and optionally one or more further antibodies may be selected from the group consisting of taxanes such as paclitaxel (Taxol), docetaxel (Taxotere) and SB-T-1214; cyclophosphamide; lapatinib; erlotinib; imatinib; pazopanib; capecitabine; cytarabine; vinorelbine; gemcitabine; anthracyclines such as daunorubicin, doxorubicin, epirubicin, idarubicin, valrubicin and mitoxantrone; aromatase inhibitors such as aminoglutethimide, testolactone (Teslac), anastrozole (Arimidex), letrozole (Femara), exemestane (Aromasin), vorozole (Rivizor), formestane (Lentaron), fadrozole (Afema), 4-hydroxyandrostenedione, 1,4,6-androstatrien-3,17-dione (ATD) and 4-androstene-3,6,17-trione (6-OXO); topoisomerase inhibitors such as irinotecan, topotecan, camptothecin, lamellarin D, etoposide (VP-16), teniposide, doxorubicin, daunorubicin, mitoxantrone, amsacrine, ellipticines, aurintricarboxylic acid and HU-331; platinum based chemotherapeutic agents such as cis-diamminedichloroplatinum(II) (cisplatin), cis-diammine(1,1-cyclobutanedicarboxylato)platinum(II) (carboplatin) and [(1R,2R)-cyclohexane-1,2-diamine](ethanedioato-O,O′)platinum(II) (oxaliplatin), and antimetabolites, in particular antifolates such as methotrexate, pemetrexed, raltitrexed and pralatrexate, pyrimidine analogues such as fluoruracil, gemcitabine, floxuridine, 5-fluorouracil and tegafur-uracil, and purine analogues. Treatment with the humanized anti-CD40 antibody may further be combined with immunostimulatory agents, cytokines, chemokines, radiation therapy, vaccines such as protein, peptide or RNA vaccines, B-Raf inhibitors such as vemurafenib, dexametasone, protease inhibitors such as bortezomib, and lenalidomide.

For use in the treatment of cancer wherein the cells express CD40, the humanized antibody may be coupled to a further agent as described above, wherein the further agent preferably is a cytotoxic agent such as a radionuclide or a cytotoxin. One or more of the anti-cancer therapeutic agents described above may also be used as further agent for coupling to the humanized anti-CD40 antibody. Furthermore, the humanized antibody may be engineered so as to enhance its ability to activate the patient's immune response, in particular the ability to activate ADCC (antibody-dependent cell-mediated cytotoxicity) and/or CDC (complement dependent cytotoxicity). For example, this may be achieved by optimizing the amino acid sequence and/or the glycosylation pattern of the antibody, in particular of its constant regions.

For use as detection agent in diagnosis, prognosis and/or monitoring of a disease, the humanized antibody preferably is coupled to a labeling agent which is capable of producing a detectable signal. In particular, said labeling agent may be a radionuclide, a fluorophore or an enzyme.

### FIGURES

FIG. 1 shows the Coomassie blue stained gel of an isoelectric focusing assay with chimeric and humanized anti-CD40 antibodies. Lane 1: chimeric IgG1 produced in NM-H9D8; lane 2: chimeric IgG1 produced in NM-H9D8-E6Q12; lane 3: chimeric IgG2 produced in NM-H9D8-E6Q12; lane 4: chimeric IgG2 produced in NM-H9D8; lane 5: control IgG2; lane 6: chimeric IgG2 produced in CHO; lane 7: chimeric IgG1 produced in CHO; lane 8: humanized IgG1 produced in NM-H9D8-E6Q12; lane 9: humanized IgG1 produced in NM-H9D8; lane 10: humanized IgG2 produced in NM-H9D8-E6Q12; lane 11: humanized IgG2 produced in NM-H9D8; lane 12: marker proteins with known isoelectric point.

FIG. 2 shows anti-CD40 antibody binding to CD40 positive Raji cells. The mean fluorescence intensity of labeled anti-CD40 antibodies on Raji cells was determined in a flow cytometric analysis dependent on the antibody concentration. Chimeric (cLob) and humanized (hLob) anti-CD40 antibodies produced in NM-H9D8 and chimeric ChiLob 7/4 produced in CHO were used as IgG1-type (A) and as IgG2-type (B) antibody.

FIG. 3 shows anti-CD40 antibody binding to Fcγ receptor IIA (A, B), IIB (C, D) and IIIA (E, F). Chimeric (cLob) and humanized (hLob) anti-CD40 antibodies produced in NM-H9D8 or NM-H9D8-E6-Q12 were used as IgG1-type (A, C, E) and as IgG2-type (B, D, F) antibody.

FIG. 4 shows the results of NFκB activation assays using the CD40L transfected HEK-Blue reporter cell line after stimulation with humanized anti-CD40 antibodies (hLob) produced in NM-H9D8 or NM-H9D8-E6-Q12 as IgG2-type antibody (A). A comparative humanized anti-CD40 antibody with lower CD40 binding affinity (hLob1), the parental anti-CD40 antibody (ChiLob 7/4), an unrelated IgG2 antibody (isotype control) and CD40L were used as controls. Comparison of NFκB activation between the humanized anti-CD40 antibody as IgG1-type or IgG2-type antibody produced in NM-H9D8 (high fucosylation) or NM-H9D8-E6-Q12 (low fucosylation) with or without cross-linking by a human NK cell line is shown (B).

FIG. 5 shows B cell proliferation induced by anti-CD40 antibody variants. Chimeric (cLob) and humanized (hLob, variant with high CD40 affinity, and hLob1, variant with lower CD40 affinity) anti-CD40 antibodies produced in NM-H9D8 and parental chimeric antibody (ChiLob 7/4) produced in CHO were used as IgG2-type antibody.

FIG. 6 shows the expression of the activation and maturation markers CD80 (A), CD86 (B), HLA-DR (C) and CD83 (D) on the surface of human dendritic cells after stimulation by humanized anti-CD40 antibodies (hLob) produced in NM-H9D8 or NM-H9D8-E6-Q12 as IgG1-type antibody. Expression was analyzed by flow cytometry, MFI (A-C) or percentage of positive cells (D) within the CD11c highly positive DC population.

FIG. 7 shows the results of a mixed leukocyte reaction with primary human dendritic cells, T cells and humanized (hLob) anti-CD40 antibodies produced in NM-H9D8 or NM-H9D8-E6-Q12 as IgG1-type antibody. Expression of the activation marker CD25 (A) and the costimulatory molecule CD137 (B) was analyzed by flow cytometry and is shown as the percentage of positive cells within the CD8 positive T cell population.

### EXAMPLES

### Example 1: Humanization of the Murine Heavy and Light Chain Variable Regions of an Anti-CD40 Antibody

The nucleic acid sequences coding for the murine heavy and light chain variable regions (VH, SEQ ID NO: 4, and VL, SEQ ID NO: 8) of a monoclonal agonistic anti-CD40 antibody were ligated to the genomic sequences of the human constant γ1 or γ2 region (CH) and the human constant κ region (CL), respectively.

On the basis of these chimeric clones, humanized antibodies were constructed. To this end, point mutations were introduced into the nucleic acid sequences of the murine framework regions of VH and VL in order to generate the corresponding human framework regions. The target human framework regions were selected from a human germ line antibody library. In particular, the most related framework regions were chosen from the library depending on their overall sequence similarity and their CDR loop classification. All data obtained were considered to design a set of different variable sequences of humanized variable light and variable heavy chains. Some of the variants contain back-mutations to the murine sequence on critical positions. The humanized variants of the light chain variable region were cloned in a κ-chain vector and the humanized variants of the heavy chain variable region were cloned in a γ1- or a γ2-chain vector.

By the above described method, the following humanized antibody heavy and light chains variable regions were obtained.

TABLE 1

| heavy chain | | light chain | |
|---|---|---|---|
| variable region | SEQ ID | variable region | SEQ ID |
| mVH | 22 | mVL | 23 |
| VH1 | 1 | VL1 | 15 |
| VH2 | 2 | VL2 | 16 |
| VH3 | 3 | VL3 | 17 |
| VH4 | 4 | | |
| VH5 | 5 | | |
| VH6 | 6 | | |
| VH7 | 7 | | |
| VH8 | 8 | | |
| VH9 | 9 | | |
| VH10 | 10 | | |

mVH and mVL represent the murine heavy and light chain variable regions, respectively, which were used as basis for the humanization.

### Example 2: Determining the Isoelectric Point of the Humanized Antibody Variants

Isoelectric focusing is an analytical method for separation of proteins according to their isoelectric point by electrophoresis in polyacrylamide gels containing carrier ampholytes that result in a pH gradient. Thereby, different glycoforms of the proteins result in distinctive band patterns.

For IEF analyses, buffer of the antibody preparations was exchanged to nanopure water. Antibody proteins were applied to pH 6-9 precast horizontal gels and run on a Multiphor II instrument (GE Healthcare). Afterwards, gels were stained using Coomassie brilliant blue (0.02% Coomassie brilliant blue G250; 5% aluminum sulfate hydrate; 10% ethanol; 2% ortho-phosphoric acid).

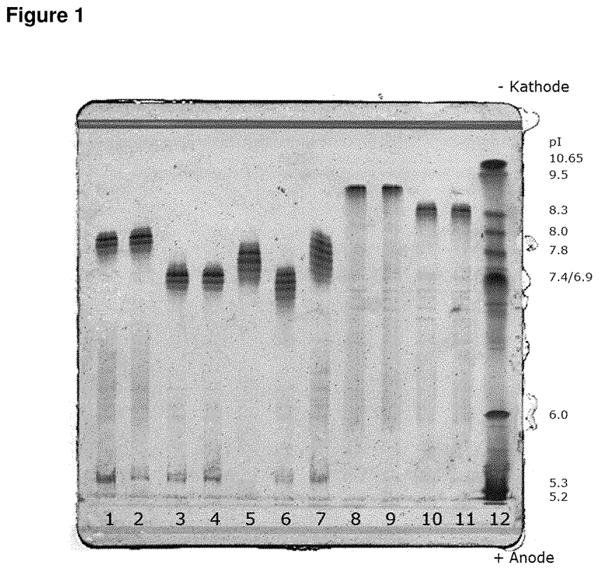

FIG. 1 shows an example of an IEF gel of cLob and hLob samples expressed in NM-H9D8 and NM-H9D8-E6Q12 compared to ChiLob7/4. There are several distinct bands to be observed for each protein. In general, for all IgG1 proteins, the isoform pattern is shifted to more basic forms compared to all IgG2 antibodies. hLob isoforms have a more basic isoelectric point (pI) than the cLob isoforms. Chilob 7/4 IgG1 contains more bands (isoforms) than ChiLob 7/4 IgG2. These general differences for the observed pIs correlate with the theoretical isoelectric points determined for the pure amino acid sequence of the antibodies not considering the attached glycan structures (see FIG. 1).

### Example 3: Binding of the Humanized Antibody Variants to Immobilized CD40

Following expression of the different constructs in NM-H9D8 and NM-H9D8-E6Q12 cells, the titer of the humanized antibody variants was determined and their concentration adjusted. Then, the humanized antibodies were screened in an antigen ELISA and via surface plasmon resonance. All variants showed significant binding to CD40 similar to that of the parental chimeric antibody. In particular the variant VH10/VL2 showed good results with a dissociation constant K_{D} for binding to CD40 of 25 nM, which was even slightly lower (i.e. the binding is stronger) than that of the chimeric anti-CD40 antibody being 28 nM.

### Example 4: Binding of the Humanized Antibody Variants to Different Cells Expressing CD40

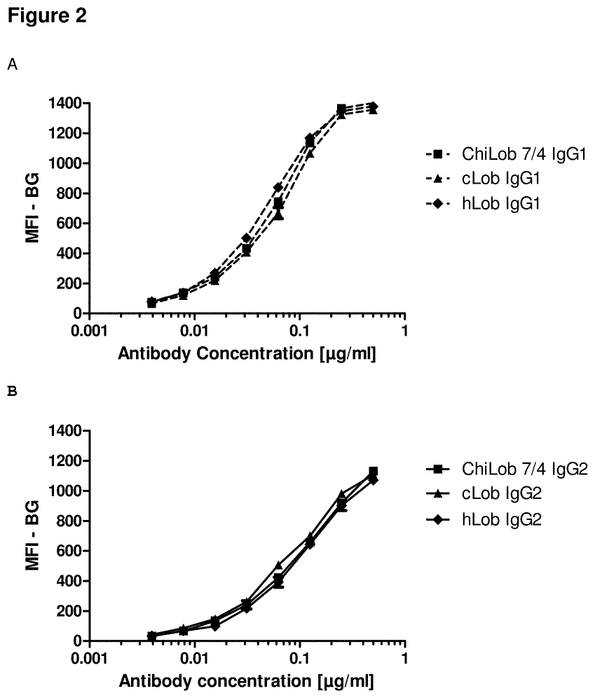

Using the best heavy and light chain variable region variants (VH10/VL2), IgG1 and IgG2 antibodies were produced and a flow cytometric analysis with CD40-positive Raji cells was performed. The binding of the humanized antibodies and their chimeric counterparts to the cells is shown in FIG. 2. It was demonstrated that the humanized antibodies have antigen binding properties comparable to those of the chimeric antibody from which they are derived.

### Example 5: Binding of the Humanized Antibody Variants to Fcγ Receptors IIA and IIB

FcγR binding assays for FcγRIIIa (CD16a), FcγRIIa (CD32a) and FcγRIIb (CD32b) are based on the AlphaScreen® technology of PerkinElmer. The AlphaScreen® platform relies on simple bead-based technology of PerkinElmer and is a more efficient alternative to traditional ELISA.

For the receptor binding assays, a His-tagged FcγR (FcγRIIIa: Glycotope GmbH, FcgRIIa: His-tagged recombinant human CD32a, Sino Biological, FcgRIIb: His-tagged recombinant human CD32b, Sino Biological) is captured by Ni-chelate donor beads.

Anti-CD40 antibodies and rabbit-anti-mouse coupled acceptor beads compete for binding to FcγR. In case of interaction of FcγR with rabbit-anti-mouse acceptor beads, donor and acceptor beads come into close proximity which leads, upon laser excitation at 680 nm, to light emission. A maximum signal is achieved (signalₘₐₓ) without a competitor. In case of competition, where a test antibody binds to FcγR, the signalₘₐₓ is reduced in a concentration-dependent manner. Chemiluminescence was quantified by measurement at 520-620 nm (AlphaScreen® method) using an EnSpire 2300 multilabel reader (PerkinElmer). All results were expressed as the mean±standard deviation of duplicate samples. The data were evaluated and calculated using non-linear curve fitting (sigmoidal dose-response variable slope) with GraphPad Prism 5 software. As a result, a concentration dependent sigmoidal curve was obtained, which is defined by top-plateau, bottom-plateau, slope and EC50.

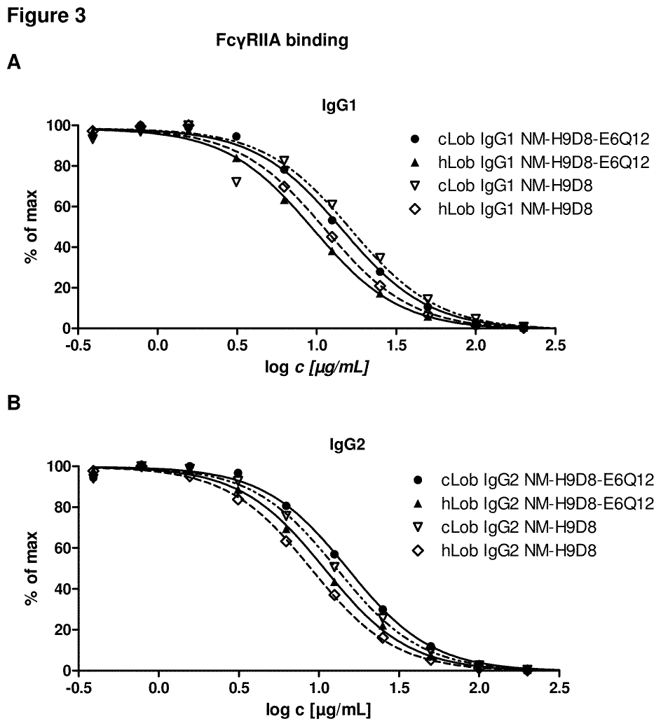

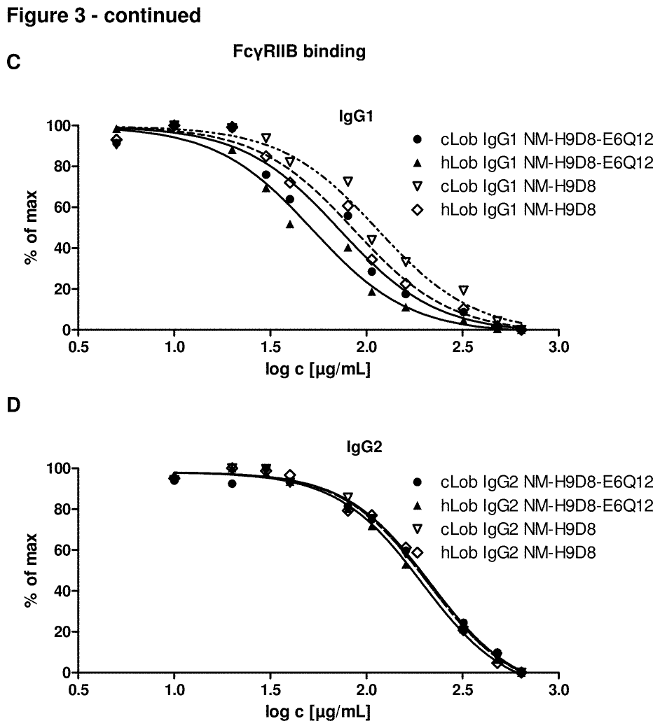

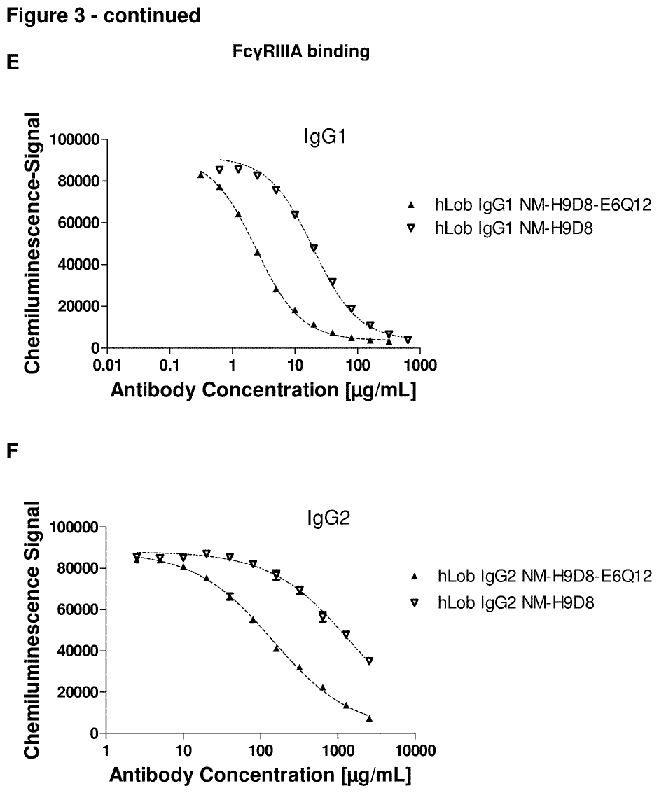

While the FcγR IIA binding affinity was comparable for IgG1 and IgG2 antibodies, hLob showed higher FcγR IIA binding than cLob (FIGS. 3A and B).

IgG1 antibody variants expressed in NM-H9D8-E6Q12 bound with higher affinity to FcγR IIB and hLob showed higher FcγR IIB binding than cLob (FIG. 3C).

On the other hand, IgG2 antibody variants bound with lower affinity to FcγRIIB than IgG1 antibody variants, but the glycovariants tested showed comparable FcγRIIB binding (FIG. 3D).

IgG1 and IgG2 antibody variants expressed in NM-H9D8-E6Q12 bound with higher affinity to FcγR IIIA (FIGS. 3E and F).

### Example 6: NFκB Activation in a CD40L Transfected Reporter Cell Line Induced by the Humanized Antibody Variants

NFκB activation is a key marker in the signal transduction cascade activated by agonistic engagement of CD40 on the cell surface. In order to measure NFκB activation, a CD40L-transfected HEK-Blue sensor cell line (Invivogen) was used. Upon NFκB activation following CD40 stimulation, these cells secrete embryonic alkaline phosphatase that is measured by QUANTI-Blue (a SEAP detection assay by Invivogen).

Briefly, HEK-Blue CD40L cells were incubated with anti-CD40 antibodies, CD40L (as positive control) or isotype controls (negative control) at a concentration of 100 ng/ml for one day and SEAP levels were measured in the supernatant by the QUANTI-Blue detection assay.

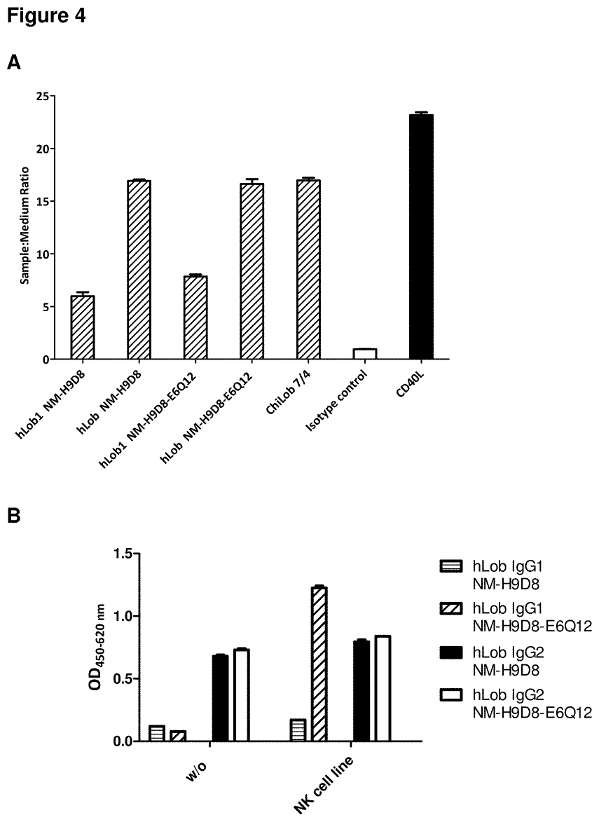

FIG. 4A shows the results of the incubation with anti-CD40 antibodies of the IgG2 isotype. The humanized hLob antibodies of the variant VH10/VL2 showed similar NFκB activation as the parental and chimeric antibodies. On the contrary, the reference humanization with lower affinity (hLob1) resulted in a significantly reduced NFκB activation.

NFκB activation mediated by anti-CD40 antibodies of the IgG1 isotype strongly depended on the cross-linking of the antibodies while IgG2 antibodies showed NFκB activation independent of the presence of crosslinking agents. The addition of a NK cell line expressing FcgRIIIA as a crosslinker showed a stronger increase in NFκB activation mediated by humanized anti-CD40 antibodies expressed in NM-H9D8-E6Q12 than by humanized anti-CD40 antibodies expressed in NM-H9D8 (FIG. 4B).

### Example 7: Induction of B Cell Proliferation by Humanized Anti-CD40 Antibody Variants

B cell proliferation is induced by binding of agonistic anti-CD40 antibodies to primary human B cells.

Briefly, human PBMC were thawed and B cell enrichment was performed using Dynabeads for untouched human B cells (Thermo Fisher). B cells were cultured for several days in the presence of humanized anti-CD40 antibodies (hLob), chimeric antibodies cLob, and ChiLob 7/4 or CD40L as a positive control. ATP content in the samples was measure as a marker for viable cells using the CellTiter-Glo assay (Promega).

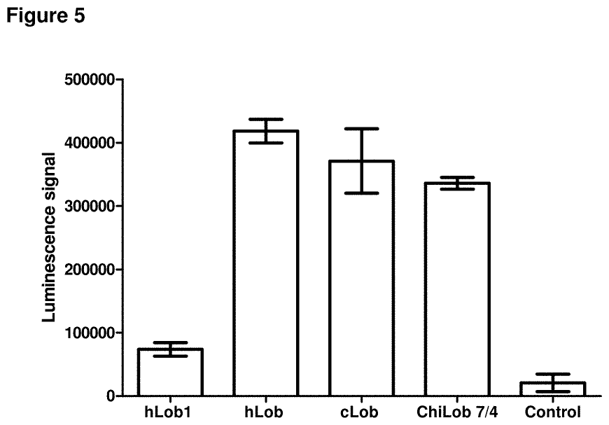

FIG. 5 shows the results of a B cell proliferation assay using anti-CD40 antibody variants of the IgG2 isotype at a concentration of 100 ng/ml after an incubation time of 8 days. The humanized antibody with the lower anti-CD40 binding affinity showed a significantly decreased induction of B cell proliferation.

### Example 8: Increased Stimulation of Dendritic Cells by Anti-CD40 Antibody Variants

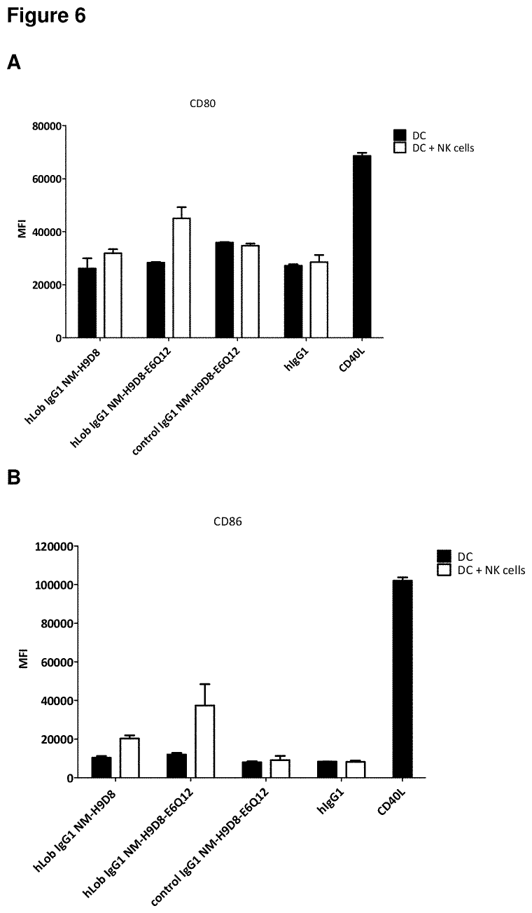

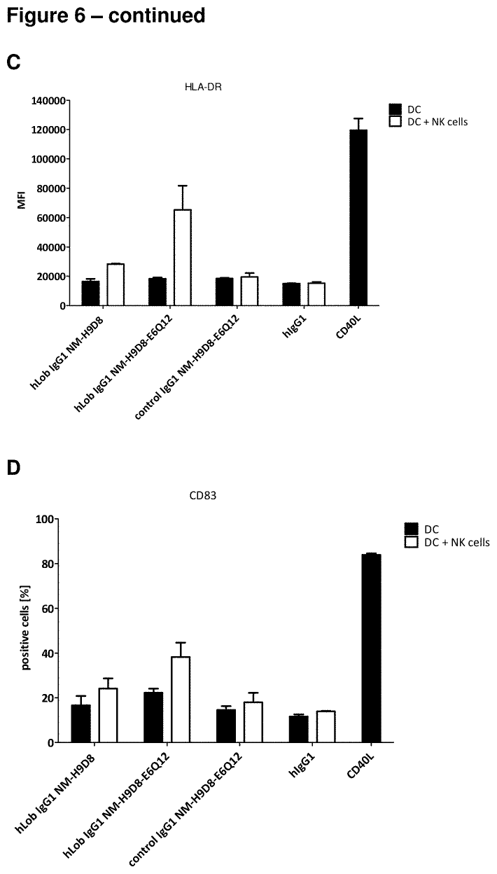

Primary human PBMC were isolated from buffy coats of healthy donors. Monocytes were isolated from half of the PBMC preparation using the Dynabeads untouched human monocytes kit (Thermo Fisher) according to manufacturer's protocol. Monocytes were cultured for 6-7 days with RPMI medium containing 10% fetal calf serum, 50 ng/ml IL-4 and 150 ng/ml GM-CSF differentiating into immature dendritic cells (iDC). The remaining PBMCs were stored frozen for 1 week and used for the isolation of human NK cells using the Dynabeads Untouched human NK cells kit (Thermo Fisher) according to manufacturer's protocol. Thereafter, 2×10⁵ differentiated iDC were incubated for 2 days with or without 1×10⁵ NK cells in the presence of humanized anti-CD40 antibodies of the IgG1 isotype (hLob) produced in NM-H9D8 or NM-H9D8-E6Q12 (300 ng/ml) or a unspecific control antibody produced in NM-H9D8-E6Q12 or human IgG1 as negative controls in the presence of 75 ng/ml recombinant human TNF-α. Incubation with recombinant CD40L served as a positive control. The cells were harvested and analyzed by flow cytometry using directly fluorescence-labeled antibodies specific for the activation markers CD80, CD86 and HLA-DR and the maturation marker CD83. In FIG. 6, results are given as mean fluorescence intensity (A-C) or percentage of positive cells within the highly CD11c positive DC population.

In the presence of NK cells, the humanized anti-CD40 antibodies (hLob) produced in NM-H9D8-E6Q12 induced stronger activation and maturation of the dendritic cells as compared to the humanized anti-CD40 antibodies (hLob) produced in NM-H9D8 indicating that the stronger crosslinking ability of hLob produced in NM-H9D8-E6Q12 results in stronger DC activation.

### Example 9: Increased T Cell Stimulation by Humanized Anti-CD40 Antibody Variants in Mixed Lymphocyte Reactions

In mixed lymphocyte reactions, the influence of the stronger DC activation on the T cell activation was analyzed. Briefly, human monocytes were isolated from buffy coats-derived PBMCs using the Pan Monocyte Isolation Kit (Miltenyi Biotec GmbH). Monocytes were differentiated into immature dendritic cells by culturing for 7 days in MEM medium containing 20% FCS, 10% conditioned Medium, 500 U/ml IL-4 and 250 U/ml GM-CSF.

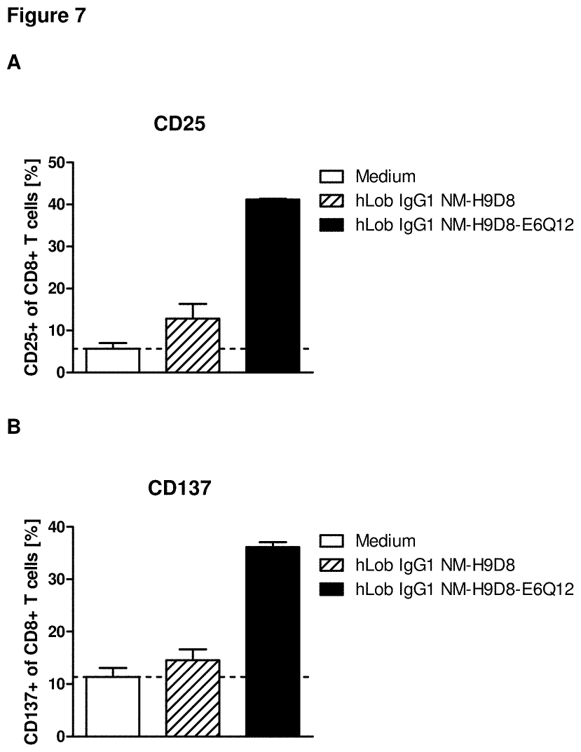

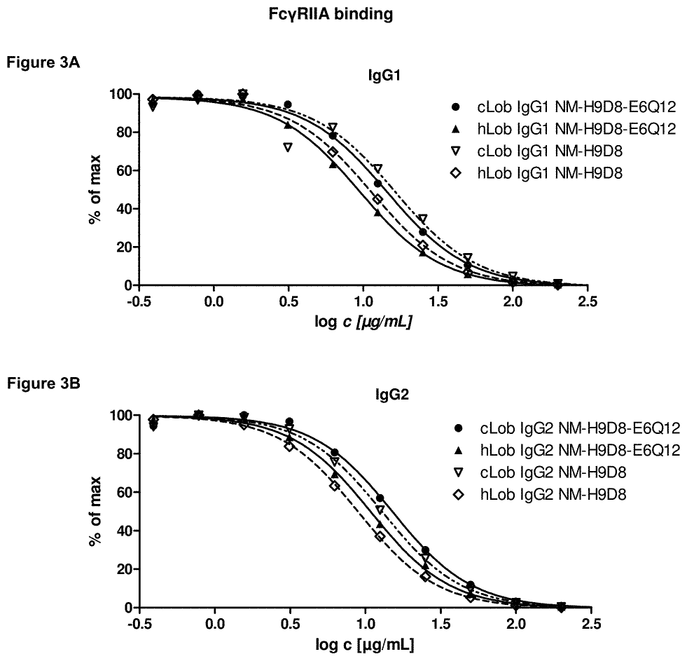

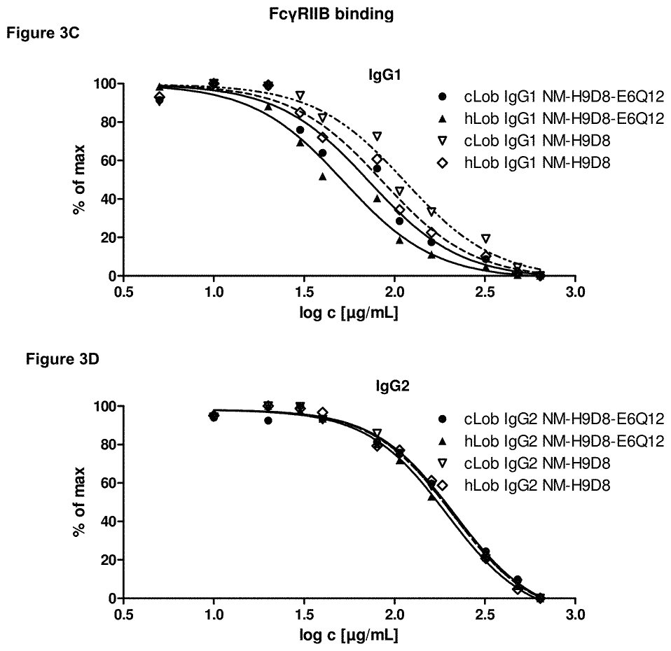

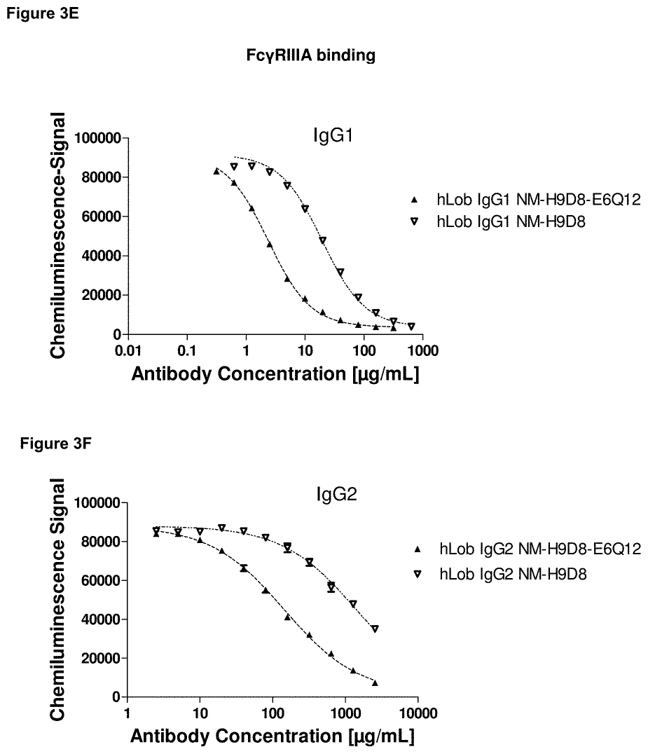

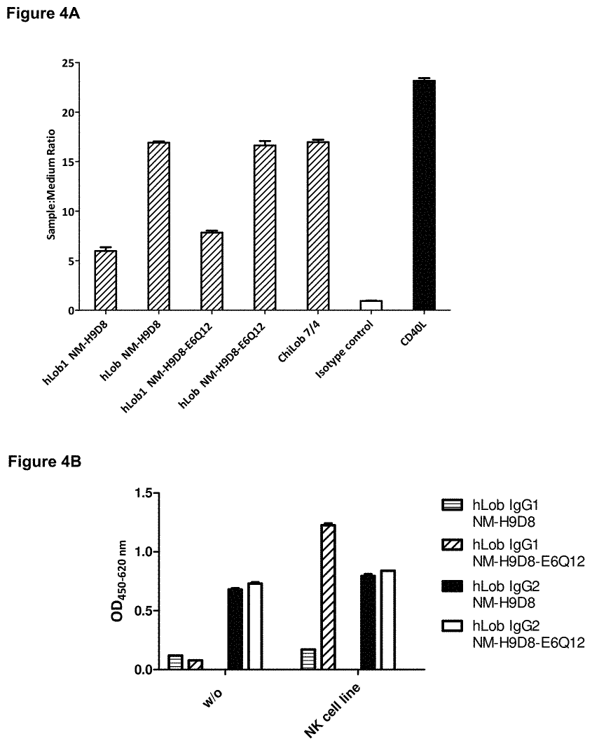

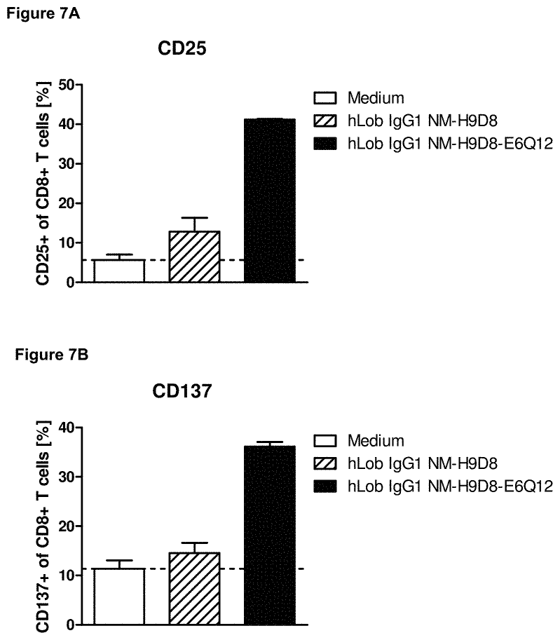

T cells were isolated from human PBMCs using the Dynabeads untouched human T Cells (Thermo Fisher). 1×10⁶ T cells were then incubated with 1×10⁵ iDC and humanized anti-CD40 antibodies of the IgG1 isotype (hLob) produced in NM-H9D8 or NM-H9D8-E6Q12 (1 μg/ml). After 5 days, activation of T cells was measured by flow cytometry analysis of the cell surface expressed activation marker CD25 in the subpopulation of cytotoxic CD8+ positive T cells (FIG. 7A). The humanized anti-CD40 antibodies (hLob) produced in NM-H9D8-E6Q12 induced stronger T cell activation than the humanized anti-CD40 antibodies (hLob) produced in NM-H9D8 indicating that the increased DC activation indeed translates into a better T cell activation. Furthermore, CD8 T cells incubated with hLob produced in NM-H9D8-E6Q12 showed stronger expression of CD137 (4-1 BB), a T-cell costimulatory molecule (FIG. 7B).

### Identification of the Deposited Biological Material

The cell lines DSM ACC 2806, DSM ACC 2807 and DSM ACC 2856 were deposited at the DSMZ—Deutsche Sammlung von Mikroorganismen and Zellkulturen GmbH, Inhoffenstraße 7B, 38124 Braunschweig (DE) by Glycotope GmbH, Robert-Rössle-Str. 10, 13125 Berlin (DE) on the dates indicated in the following table.

| Name of the | Accession | | Date of |
|---|---|---|---|
| Cell Line | Number | Depositor | Deposition |
| NM-H9D8 | DSM ACC 2806 | Glycotope | Sep. 15, 2006 |
| | | GmbH | |
| NM-H9D8-E6 | DSM ACC 2807 | Glycotope | Oct. 5, 2006 |
| | | GmbH | |
| NM-H9D8- | DSM ACC 2856 | Glycotope | Aug. 8, 2007 |
| E6Q12 | | GmbH | |

1. A humanized antibody which is capable of binding to CD40 and which comprises a heavy chain variable region, wherein the heavy chain variable region comprises the amino acid sequence of SEQ ID NO: 11, or an amino acid sequence which is at least 90% identical to the amino acid sequence of SEQ ID NO: 11.

2. The antibody according to claim 1, wherein the heavy chain variable region comprises the complementarity determining regions CDR-H1 having the amino acid sequence of SEQ ID NO: 12, CDR-H2 having the amino acid sequence of SEQ ID NO: 13, and CDR-H3 having the amino acid sequence of SEQ ID NO: 14.

3. The antibody according to claim 2, wherein the heavy chain variable region comprises an amino acid sequence selected from the group of SEQ ID NOs: 1 to 10.

4. The antibody according to claim 1, wherein the antibody comprises a light chain variable region, wherein the light chain variable region comprises the amino acid sequence of SEQ ID NO: 18, or an amino acid sequence which is at least 90% identical to the amino acid sequence of SEQ ID NO: 18.

5. The antibody according to claim 4, wherein the light chain variable region comprises the complementarity determining regions CDR-L1 having the amino acid sequence of SEQ ID NO: 19, CDR-L2 having the amino acid sequence of SEQ ID NO: 20, and CDR-L3 having the amino acid sequence of SEQ ID NO: 21.

6. The antibody according to claim 5, wherein the light chain variable region comprises an amino acid sequence selected from the group of SEQ ID NOs: 15 to 17.

7. The antibody according to claim 4, wherein the heavy chain variable region comprises the amino acid sequence of SEQ ID NO: 10 and the light chain variable region comprises the amino acid sequence of SEQ ID NO: 16.

8. The antibody according to claim 1, wherein the antibody comprises an Fc region.

9. The antibody according to claim 8, wherein the antibody is an IgG1-type, IgG2-type or IgG4-type antibody.

10. The antibody according to claim 8, wherein the antibody comprises a glycosylation pattern at the Fc region having one or more of the following characteristics:
(i) a detectable amount of glycans carrying a bisecting GlcNAc residue;
(ii) a relative amount of glycans carrying at least one sialic acid residue of at least 2% of the total amount of glycans attached to the Fc glycosylation sites of the antibody in a composition.

11. The antibody according to claim 8, wherein the antibody has a binding affinity towards human Fcγ receptor IIA and/or human Fcγ receptor IIB which is stronger than that of an antibody having the same constant regions and wherein the heavy chain variable region has the amino acid sequence of SEQ ID NO: 22 and the light chain variable region has the amino acid sequence of SEQ ID NO: 23.

12. The antibody according to claim 1, obtainable by production in a human cell line selected from the group consisting of NM-H9D8 (DSM ACC 2806), NM-H9D8-E6 (DSM ACC 2807), NM-H9D8-E6Q12 (DSM ACC 2856) and cell lines derived therefrom.

13. A nucleic acid encoding the antibody according to claim 1.

14. An expression cassette or vector comprising the nucleic acid according to claim 13 and a promoter operatively connected with said nucleic acid.

15. A host cell comprising the nucleic acid according to claim 13.

16. A conjugate comprising the antibody according to claim 1 conjugated to a further agent.

17. The conjugate according to claim 16, wherein the further agent is a cytotoxic agent, tumor-specific antibody or immune checkpoint blocking or activating antibody.

18-19. (canceled)

20. A pharmaceutical composition comprising the antibody according to claim 1, and a carrier, diluent, or pharmaceutically-acceptable excipient.

21. A method for treating a cancer, an infection, or an immunodeficiency disorder, comprising administering the composition of claim 20 to a subject in need thereof.

22. The method according to claim 21, wherein said method is a method for treating a cancer, and wherein the cancer is selected from the group consisting of ovarian cancer, breast cancer, pancreatic cancer, lung cancer, leukemia and lymphoma, especially chronic lymphatic leukemia, non-Hodgkin lymphoma, diffuse large B-cell lymphoma and B cell malignancies.


SEQUENCE LISTING (23 sequences)

SEQ ID NO: 1 - protein, 122 aa, Artificial Sequence
  Synthetic sequence, heavy chain variable region
QVQLVQSGAEVKKPGASVKVSCKASGYTFTEYIMHWVRQAPGQGLEWMGGIIPNNGGTSYNQKFKDRVTMTRDTSISTAYMELSRLRSDDTAVYYCARREVYGRNYYALDYWGQGTLVTVSS

SEQ ID NO: 2 - protein, 122 aa, Artificial Sequence
  Synthetic sequence, heavy chain variable region
QVQLVQSGAEVKKPGASVKVSCKASGYTFTEYIMHWVRQSPGQSLEWMGGIIPNNGGTSYNQKFKDRVTMTVDTSISTAYMELSRLRSDDTAVYYCARREVYGRNYYALDYWGQGTLVTVSS

SEQ ID NO: 3 - protein, 122 aa, Artificial Sequence
  Synthetic sequence, heavy chain variable region
QVQLVQSGAEVKKPGASVKVSCKASGYTFTEYIMHWVKQSPGKSLEWMGGIIPNNGGTSYNQKFKDRVTMTVDKSISTAYMELSRLRSDDTAVYYCARREVYGRNYYALDYWGQGTLVTVSS

SEQ ID NO: 4 - protein, 122 aa, Artificial Sequence
  Synthetic sequence, heavy chain variable region
QVQLVQSGPEVKKPGASVKVSCKTSGYTFTEYIMHWVRQSPGQSLEWMGGIIPNNGGTSYNQKFKDRVTMTVDKSISTAYMELSRLRSDDTAVYYCARREVYGRNYYALDYWGQGTLVTVSS

SEQ ID NO: 5 - protein, 122 aa, Artificial Sequence
  Synthetic sequence, heavy chain variable region
EVQLVQSGPEVKKPGASVKISCKTSGYTFTEYIMHWVKQSPGKSLEWIGGIIPNNGGTSYNQKFKDRVTMTVDKSISTAYMELSRLRSDDTAVYYCARREVYGRNYYALDYWGQGTLVTVSS

SEQ ID NO: 6 - protein, 122 aa, Artificial Sequence
  Synthetic sequence, heavy chain variable region
EVQLVQSGAEVKKPGASVKISCKTSGYTFTEYIMHWVKQSPGKSLEWIGGIIPNNGGTSYNQKFKDKATMTVDKSISTGYMELSRLRSDDTAVYYCTRREVYGRNYYALDYWGQGTLVTVSS

SEQ ID NO: 7 - protein, 122 aa, Artificial Sequence
  Synthetic sequence, heavy chain variable region
EVQLVQSGAEVKKPGASVKISCKTSGYTFTEYIMHWVKQSPGKSLEWIGGIIPNNGGTSYNQKFKDKATMTVDKSISTAYMELSRLRSDDTAVYYCARREVYGRNYYALDYWGQGTLVTVSS

SEQ ID NO: 8 - protein, 122 aa, Artificial Sequence
  Synthetic sequence, heavy chain variable region
EVQLVQSGAEVKKPGASVKLSCKTSGYTFTEYIMHWVKQSPGKSLEWIGGIIPNNGGTSYNQKFKDKATMTVDKSISTAYMELSRLRSDDTAVYYCARREVYGRNYYALDYWGQGTLVTVSS

SEQ ID NO: 9 - protein, 122 aa, Artificial Sequence
  Synthetic sequence, heavy chain variable region
EVQLVQSGAEVKKPGASVKISCKTSGYTFTEYIMHWVKQSPGKSLEWIGGIIPNNGGTSYNQKFKDKATMTADTSISTGYMELSRLRSDDTAVYYCTRREVYGRNYYALDYWGQGTLVTVSS

SEQ ID NO: 10 - protein, 122 aa, Artificial Sequence
  Synthetic sequence, heavy chain variable region
EVQLVQSGAEVKKPGASVKISCKTSGYTFTEYIMHWVKQSPGKSLEWIGGIIPNNGGTSYNQKFKDRVTMTVDKSISTGYMELSRLRSDDTAVYYCTRREVYGRNYYALDYWGQGTLVTVSS

SEQ ID NO: 11 - protein, 122 aa, Artificial Sequence
  Synthetic sequence, heavy chain variable region
  MISC_FEATURE (1)..(1): Xaa is Gln or Glu
  MISC_FEATURE (9)..(9): Xaa is Pro or Ala
  MISC_FEATURE (20)..(20): Xaa is Val or Ile or Leu
  MISC_FEATURE (24)..(24): Xaa is Ala or Thr
  MISC_FEATURE (38)..(38): Xaa is Arg or Lys
  MISC_FEATURE (40)..(40): Xaa is Ala or Ser
  MISC_FEATURE (43)..(43): Xaa is Gln or Lys
  MISC_FEATURE (44)..(44): Xaa is Gly or Ser
  MISC_FEATURE (48)..(48): Xaa is Met or Ile
  MISC_FEATURE (67)..(67): Xaa is Arg or Lys
  MISC_FEATURE (68)..(68): Xaa is Val or Ala
  MISC_FEATURE (72)..(72): Xaa is Arg or Ala or Val
  MISC_FEATURE (74)..(74): Xaa is Thr or Lys
  MISC_FEATURE (79)..(79): Xaa is Ala or Gly
  MISC_FEATURE (97)..(97): Xaa is Ala or Thr
XVQLVQSGXEVKKPGASVKXSCKXSGYTFTEYIMHWVXQXPGXXLEWXGGIIPNNGGTSYNQKFKDXXTMTXDXSISTXYMELSRLRSDDTAVYYCXRREVYGRNYYALDYWGQGTLVTVSS

SEQ ID NO: 12 - protein, 5 aa, Artificial Sequence
  Synthetic sequence, heavy chain CDR-H1
EYIMH

SEQ ID NO: 13 - protein, 17 aa, Artificial Sequence
  Synthetic sequence, heavy chain CDR-H2
GIIPNNGGTSYNQKFKD

SEQ ID NO: 14 - protein, 13 aa, Artificial Sequence
  Synthetic sequence, heavy chain CDR-H3
REVYGRNYYALDY

SEQ ID NO: 15 - protein, 108 aa, Artificial Sequence
  Synthetic sequence, light chain variable region
DIQMTQSPSSLSASVGDRVTITCSASQGINNYLNWYQQKPGKAPKLLIYYTSSLHSGVPSRFSGSGSGTDFTFTISSLQPEDIATYYCQQYSNLPYTFGQGTKLEIKR

SEQ ID NO: 16 - protein, 108 aa, Artificial Sequence
  Synthetic sequence, light chain variable region
DIQMTQSPSSLSASVGDRVTITCSASQGINNYLNWYQQKPGKAPKLLIYYTSSLHSGVPSRFSGSGSGTDYTFTISSLQPEDIATYYCQQYSNLPYTFGQGTKLEIKR

SEQ ID NO: 17 - protein, 108 aa, Artificial Sequence
  Synthetic sequence, light chain variable region
DIQMTQSPSSLSASVGDRVTITCSASQGINNYLNWYQQKPDGAPKLLIYYTSSLHSGVPSRFSGSGSGTDFTFTISSLEPEDIATYYCQQYSNLPYTFGQGTKLEIKR

SEQ ID NO: 18 - protein, 108 aa, Artificial Sequence
  Synthetic sequence, light chain variable region
  MISC_FEATURE (41)..(41): Xaa is Gly or Asp
  MISC_FEATURE (42)..(42): Xaa is Lys or Gly
  MISC_FEATURE (71)..(71): Xaa is Phe or Tyr
  MISC_FEATURE (79)..(79): Xaa is Gln or Glu
DIQMTQSPSSLSASVGDRVTITCSASQGINNYLNWYQQKPXXAPKLLIYYTSSLHSGVPSRFSGSGSGTDXTFTISSLXPEDIATYYCQQYSNLPYTFGQGTKLEIKR

SEQ ID NO: 19 - protein, 11 aa, Artificial Sequence
  Synthetic sequence, light chain CDR-L1
SASQGINNYLN

SEQ ID NO: 20 - protein, 7 aa, Artificial Sequence
  Synthetic sequence, light chain CRD-L2
YTSSLHS

SEQ ID NO: 21 - protein, 9 aa, Artificial Sequence
  Synthetic sequence, light chain CDR-L3
QQYSNLPYT

SEQ ID NO: 22 - protein, 122 aa, Artificial Sequence
  Synthetic sequence, heavy chain variable region
EVQLQQSGPDLVKPGASVKISCKTSGYTFTEYIMHWVKQSHGKSLEWIGGIIPNNGGTSYNQKFKDKATMTVDKSSSTGYMELRSLTSEDSAVYYCTRREVYGRNYYALDYWGQGTLVTVSS

SEQ ID NO: 23 - protein, 108 aa, Artificial Sequence
  Synthetic sequence, light chain variable region
DIQMTQTTSSLSASLGDRVTITCSASQGINNYLNWYQQKPDGTVKLLIYYTSSLHSGVPSRFSGSGSGTDYSLTISNLEPEDIATYYCQQYSNLPYTFGGGTKLEIKS